(19) United States
(12) Patent Application Publication
(10) Pub. No.: US 2026/0218261 A1
(43) Pub. Date: Jul. 30, 2026

(54) ENZYMATIC DNA SYNTHESIS

(71) Applicant: THE J. DAVID GLADSTONE INSTITUTES, A TESTAMENTARY TRUST ESTABLISHED UNDER THE WILL OF J. DAVID, San Francisco, CA (US)

(72) Inventors: Seth Shipman, San Francisca, CA (US); Christina Palka, Soquel, CA (US)

(21) Appl. No.: 19/142,941

(22) PCT Filed: Jan. 4, 2024
(86) PCT No.: PCT/US2024/010263

§ 371 Date: Jun. 24, 2025

Related U.S. Application Data
(60) Provisional application No. 63/436,898, filed on Jan. 4, 2023.

(51) Int. Cl. C12P 19/34; C12N 9/12

(52) U.S. Cl. CPC C12P 19/34; C12N 9/1252

(57) ABSTRACT

Provided herein are methods for enzymatically producing de novo DNA molecules.

### PRIORITY

This application claims the benefit of priority to U.S. Provisional Application Ser. No. 63/436,898, filed Jan. 4, 2023, which is incorporated by reference herein as if fully set forth herein.

### INCORPORATION BY REFERENCE OF SEQUENCE LISTING

This application contains a Sequence Listing which has been submitted electronically in ST26 format and is hereby incorporated by reference in its entirety. Said ST26 file, created on Jan. 4, 2024, is named “3730220WO1.xml” and is 59,911 bytes in size.

### BACKGROUND

Despite being a mature technology, it is very difficult to synthesize a DNA strand greater than 200 nucleotides in length, and most DNA synthesis companies only offer up to 120 nucleotides. In comparison, an average protein-coding gene is of the order of 2000-3000 nucleotides, and an average eukaryotic genome numbers in the billions of nucleotides. Thus, all major gene synthesis companies today rely on variations of a ‘synthesize and stitch’ technique, where overlapping 40-60-mer fragments are synthesized and stitched together by PCR (see Young, L. et al. (2004) Nucleic Acid Res. 32, e59).

### SUMMARY

Provided herein are useful compositions and methods for producing synthetic de novo DNA molecules. One embodiment provides a method to synthesize a single strand DNA segment comprising a) contacting an AbiK and/or Abi-P2 enzyme with a nucleotide, wherein the enzyme and the nucleotide form a complex; b) removing any unbound nucleotide from a); c) building the single strand DNA segment from the 3′ end of the nucleotide bound to the enzyme/nucleotide of b) by contacting the enzyme/nucleotide of b) with another nucleotide; d) removing any unbound nucleotide from c); and e) repeating the steps of contacting and removing free nucleotides until a desired length and sequence of the single strand DNA segment is formed. In one embodiment, the enzyme is AbiK. In another embodiment the AbiK enzyme has the amino acid sequence provided by at least one of SEQ ID NO: 2, SEQ ID NO:12, SEQ ID NO: 22-29 or 70% identity SEQ ID NO: 2, SEQ ID NO:12 or SEQ ID NO: 22-39 or a Template Molding (TM) value of at least about 0.5 when compared to SEQ ID NO: 2, SEQ ID NO: 12 or SEQ ID NO: 22-39. In one embodiment, the enzyme is Abi-P2. In one embodiment, the Abi-P2 enzyme has the amino acid sequence provided in SEQ ID NO: 4, SEQ ID NO: 5, SEQ ID NO: 7, SEQ ID NO: 9, SEQ ID NO: 11 or 70% identity SEQ ID NO: 4, SEQ ID NO: 5, SEQ ID NO: 7, SEQ ID NO: 9, SEQ ID NO: 11 or a Template Molding (TM) value of at least about 0.5 when compared to one of SEQ ID NO: 4, SEQ ID NO: 5, SEQ ID NO: 7, SEQ ID NO: 9 or SEQ ID NO: 11.

In one embodiment, the nucleotides are bound to a reversible chain nucleotide terminator. In one embodiment, the reversible chain nucleotide terminator comprises at least one 3′-O-blocked reversible terminator and/or 3′-unblocked reversible terminator. In one embodiment, the reversible chain nucleotide terminator is removed after removing any unbound nucleotide and prior to contacting with another nucleotide to build the single strand DNA segment.

In one embodiment, the nucleotides are independently selected from A, T, G, C or a nucleotide analog. In one embodiment, the nucleotide analog comprises one or more non-naturally occurring nucleotides/nucleotide analogs including 5-bromouracil (5BU), fluorescent based analogs (2-aminopurine (2-AP), 3-MI, 6-MI, 6-MAP, pyrrolo-dC, furan-modified bases, d5SICS, dNaM, 2-amino-8-(2-thienyl)purine (s), pyridine-2-one (y), 7-(2-thienyl)imidazo[4,5-b]pyridine (Ds), pyrrole-2-carbaldehyde (Pa), 4-[3-(6-aminohexanamido)-1-propynyl]-2-nitropyrrole (Px); xanthine, 5-(2,4 diaminopyrimidine) or a combination thereof.

In one embodiment, the single strand DNA segment is 1 to about 10,000 nucleotides long. In another embodiment, the single strand DNA segment comprises naturally and non-naturally occurring nucleotides.

In one embodiment, the protein or nucleotide is attached to a solid substrate. In one embodiment, the solid substrate is paper, ceramic, gold, glass, metal, plastic, polystyrene, protein affinity tag resin, protein cleanup column, silicone or a combination thereof.

One embodiment provides a DNA synthesis device comprising: a reaction chamber; and the enzyme/nucleotide complex described herein, located at least partially within the reaction chamber. In one embodiment, the reaction chamber is a well, a channel, a cartridge, a or a pore. In another embodiment, the device is a flow cytometry device, a microarray, thermocycler, droplet sorter, or a 96 well plate. In one embodiment, the device is an automated device.

### BRIEF DESCRIPTION OF THE DRAWING

FIG. 1 provides a schematic of elongation of a de novo sequence of DNA.

FIG. 2 provides protein AbiK and AbiP2 sequence alignment using MUSCLE. Exact match amino acids are indicated by an “*” and amino acids that are similar are noted with a “:”.

FIGS. 3A-3C provide a structural overlay of (A) AbiK (red) and Abi-P2 (blue) (B) AbiK (red) and TdT (blue) and (C) AbiK (red) and MuLV (blue) as calculated using TM-align (4).

FIGS. 4A-4C demonstrate that protein incorporates two different reversible terminator groups.

FIG. 5 provides a schematic demonstrating that the reaction can be blocked and unblocked using reversible terminators.

### DESCRIPTION OF THE INVENTION

The methods provide herein are useful for producing synthetic de novo DNA molecules, such as those needed for RNA based vaccines, synthesis of long DNA segments de novo or DNA having one or more non-naturally occurring nucleotides.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, an “essentially pure” preparation of a particular DNA or protein is a preparation wherein at least about 90%, at least about 95%, such as at least about 99%, by weight, of the DNA or protein in the preparation. Alternatively, purity can be defined as the amount of correct DNA sequence, as at least 85%, 90%, 95%, 96%, 97%, 98%, 99%, or 100% correct DNA sequence.

A “fragment” or “segment” is a portion of a longer DNA sequence comprising at least two nucleotides. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

### Method of Enzymatic DNA Synthesis

Currently DNA is synthesized using chemical synthesis. This process produces molecules that are short (less than 60 bases) without significant increase in cost and time. Enzymatic DNA synthesis using AbiK has been shown to produce molecules 100's of bases long, very rapidly. This will result in a system with very little chemical waste and much longer DNA molecules at a significantly decreased cost.

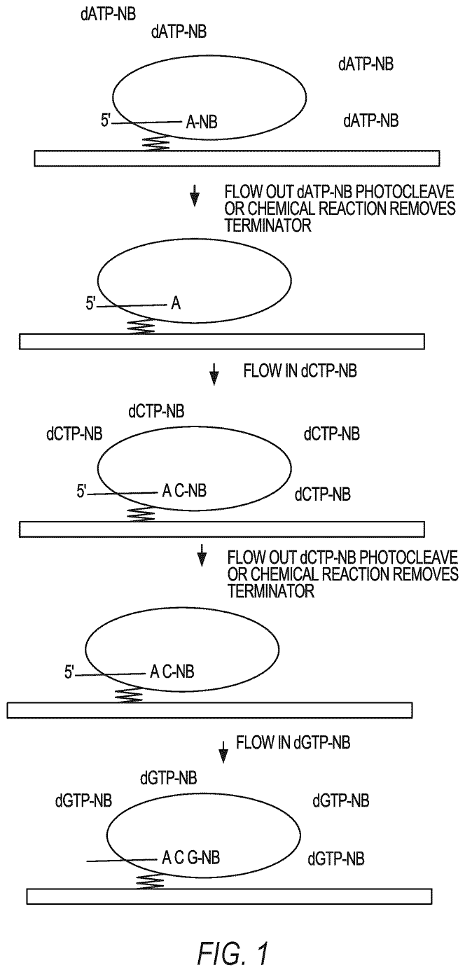

Provided herein are compositions and methods for enzymatic de novo DNA synthesis as described in FIG. 1, including Abi-family proteins for use in enzymatic DNA synthesis. AbiK, Abi-P2, proteins having 70% or greater sequence identity to AbiK or Abi-P2, proteins having a TM value of greater than about 0.5 to AbiK or Abi-P2 proteins or a combination thereof are used in a method to synthesize de novo DNA sequences (FIG. 1).

In step 1), the addition of single nucleotides onto a chain of DNA is achieved by flowing nucleotides into a flow cell. Enforcement of the addition of only a single nucleotide will be achieved using nucleotides that include a reversible chain terminator (FIG. 1 and below). In step 2), continuation of the DNA synthesis is controlled by specific wavelengths or appropriate chemical conditions that remove the chain terminator, resulting in an activated nucleotide. After the chain terminator has been removed the addition of the next nucleotide will be achieved by repeating Step 1 and Step 2.

In the flow cell setup, either the protein can be immobilized to the surface as illustrated in FIG. 1, or the DNA could be immobilized to the surface. The flow cell will move liquids efficiently in and out of the chamber with the protein and nucleic acid.

To release the DNA molecules from the enzyme/DNA complex, a restriction enzyme site can be synthesized on the end of the DNA strand and cut at the appropriate time, by, for example, flowing the enzyme into the flow cell. For example, BsaI will cut outside of its recognition site allowing for no unwanted DNA sequence to remain on the product. This will release the DNA from its protein substrate. Alternatively, the protein can be degraded by flowing in proteinase K, which will also release the DNA. Alternately the synthesized DNA can be used made double stranded, amplified, and eluted using PCR technology.

This method can produce nucleic acid segments from 1 to about 10,000 nucleotides long, including segments from about 2 to about 5,000 nucleotides long, from about 2 to about 2,000 nucleotides long, from about 2 to about 1,000 nucleotides long, including 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, 1000, 1200, 1400, 1600, 1800, 2000, 2500, 3000, 3500, 4000, 4500, 5000, 5500, 6000, 6500, 7000, 7500, 8000, 8500, 9000, 9500 or 10,000 nucleotides long.

### Reverse Transcriptase/Abi Polymerases

Companies (DNA Script, Molecular Assemblies, Nucera, Kern Systems, and Camena) have been built around enzymatic DNA synthesis. However, all these companies use the polymerase Terminal Transferase for the synthesis or DNA assembly technologies which involve using pre-synthesized fragments as starting blocks. Efforts to use this enzyme are hampered largely by an inability of polymerases to accept modified nucleotides. Provided herein is the first effort that uses reverse transcriptases, instead of polymerases, for DNA synthesis. Polymerases specialize in very precisely copying DNA, which explains their inability to utilize modified nucleotides. Reverse transcriptases on the other hand are much less discriminating and can incorporate a wide range of nucleotide modifications. This makes reverse transcriptase an ideal candidate for the use in enzymatic DNA synthesis. While herein we are calling this protein a reverse transcriptase, it is not truly a reverse transcriptase as this protein does not need to copy an RNA template. This protein is not a polymerase either as it does not copy DNA. The creation of de novo synthesized DNA (random or otherwise) does not have a clear family in biology. The protein can also be termed a de novo synthesizing DNA enzyme or de novo polymerase.

Bacteria have several anti-phage defense strategies that act at different stages of phage infection (1). Some mechanisms prevent phage entry by blocking phage absorption(?) to the cell surface or by inhibiting the injection of viral DNA into the cell. Other mechanisms target and degrade genomic DNA of the invading phage, such as restriction-modification and CRIPSR-Cas systems (2,3). Further, the bacterial cell can respond to infection using toxin-antitoxin or abortive infection systems that trigger dormancy, temporary growth arrest or cell death (4,5).

Abortive infection (Abi) is a process of programmed cell death that prevents the release of virions and thus their spread to other bacterial cells in the population. Most Abi systems that have been identified to date have been characterized in Escherichia coli and Lactococcus lactis (1). Protein effectors that are involved in these systems are usually plasmid encoded. They have a range of activities and are believed to initiate cell death through different mechanisms. Among these are three systems that involve the activity of proteins that are related to reverse transcriptases (RTs): AbiA, AbiK and Abi-P2 (8). Methods for determining Abi activity, such as AbiK, have been described (see, for example, Figel et al. Nucleic Acids Res. 2022 Sep. 23; 50(17):10026-10040. doi: 10.1093/nar/gkac772 and Wang et al. Nucleic Acids Res. 2011 Sep. 1; 39(17):7620-9. doi: 10.1093/nar/gkr397. Epub 2011 Jun. 15).

### AbiK

The AbiK system in L. lactis is encoded by a single, constitutively transcribed gene that is located on the native pSRQ800 plasmid (6), the sequence of the gene and protein it codes for are known in the art and provided herein below. The gene is also commercially available (e.g., Ll-AbiK; the synthetic gene can be purchased from BioBasic and cloned into an expression vector to produce recombinant protein that can be purified for use in the methods provided herein; ncbi.nlm.nih.gov/nuccore/U35629.2?from=3297&to=5096&strand=2).

```
SEQ ID NO: 1
ATGAAAAAAGAGTTTACTGAATTATATGATTTTATATTTGATCCTATTTTTCTTGTAAGATACGGCTATT
ATGATAGATCTATTAAAAACAAAAAAATGAATACTGCAAAAGTTGAATTAGACAATGAATATGGAAAATC
AGATTCTTTTTATTTTAAAGTATTTAATATGGAATCCTTTGCAGATTATTTAAGGAGTCATGATTTAAAA
ACACATTTTAACGGTAAAAAACCTCTATCAACAGACCCAGTATATTTTAATATTCCAAAAAATATAGAAG
CTAGAAGACAATATAAGATGCCCAATTTATACAGTTATATGGCATTAAATTATTATATATGTGACAATAA
AAAAGAGTTTATAGAAGTATTTATTGATAACAAATTTTCAACGTCAAAATTTTTTAATCAATTGAATTTT
GATTATCCTAAGACACAAGAAATTACACAAACATTATTATATGGAGGAATAAAGAAATTACATTTAGATT
TATCTAATTTTTATCATACTTTATATACACATAGTATACCATGGATGATTGATGGAAAATCTGCATCTAA
ACAAAATAGAAAAAAAGGGTTTTCTAATACATTAGATACTTTGATTACAGCTTGTCAATACGACGAAACA
CATGGCATTCCAACTGGAAATCTATTGTCTAGGATTATTACCGAACTATATATGTGCCATTTTGATAAAC
AAATGGAATATAAGAAGTTTGTGTATTCAAGATATGTAGATGATTTTATATTTCCGTTTACTTTTGAGAA
TGAAAAGCAAGAATTTTTAAATGAATTTAATCTAATCTGTCGAGAAAATAACTTAATTATTAATGATAAT
AAAACGAAAGTTGACAATTTCCCGTTTGTTGATAAATCGAGTAAATCGGATATTTTTTCTTTTTTTGAAA
ATATTACTTCAACTAATTCCAACGACAAGTGGATTAAAGAAATAAGCAATTTTATAGATTATTGTGTGAA
TGAAGAACATTTAGGGAATAAGGGAGCTATAAAATGTATTTTCCCAGTTATAACAAATACATTGAAACAA
AAAAAAGTAGATACTAAAAATATAGACAATATCTTTTCGAAAAGAAACATGGTTACCAATTTTAATGTTT
TCGAAAAAATATTAGATTTATCATTAAAAGATTCAAGATTAACTAATAAGTTTTTGACTTTCTTTGAAAA
TATTAATGAATTTGGATTTTCAAGTTTATCAGCTTCAAATATTGTAAAAAAATATTTTAGTAATAATTCA
AAGGGCTTAAAAGAAAAAATAGACCACTATCGTAAAAATAATTTTAATCAAGAATTATATCAAATATTGT
TGTATATGGTTGTCTTTGAAATAGATGATTTATTAAATCAAGAAGAATTACTAAACTTAATTGATTTAAA
TATTGATGATTATTCTTTAATTTTAGGGACGATTTTATACCTAAAGAATAGTTCATATAAATTGGAAAAA
TTATTAAAAAAAATAGATCAATTATTTATTAATACTCATGCCAACTACGACGTTAAAACTTCTCGTATGG
CAGAAAAATTATGGCTATTTCGTTATTTCTTTTATTTTTTAAATTGTAAGAATATTTTTAGTCAAAAAGA
GATAAATAGTTATTGTCAATCTCAAAACTATAATTCAGGACAGAACGGATATCAAACAGAACTTAATTGG
AATTATATTAAAGGTCAAGGGAAGGATCTTAGAGCGAATAACTTTTTTAATGAATTGATAGTAAAAGAAG
TTTGGTTAATTTCTTGTGGTGAGAACGAAGATTTCAAATATTTAAATTGA
SEQ ID NO: 2:
MKKEFTELYDFIFDPIFLVRYGYYDRSIKNKKMNTAKVELDNEYGKSDSFYFKVENMESFADYLRSHDLKTHENGKK
PLSTDPVYFNIPKNIEARRQYKMPNLYSYMALNYYICDNKKEFIEVFIDNKFSTSKFFNQLNFDYPKTQEITQTLLY
GGIKKLHLDLSNFYHTLYTHSIPWMIDGKSASKQNRKKGFSNTLDTLITACQYDETHGIPTGNLLSRIITELYMCHE
DKQMEYKKFVYSRYVDDFIFPFTFENEKQEFLNEFNLICRENNLIINDNKTKVDNFPFVDKSSKSDIFSFFENITST
NSNDKWIKEISNFIDYCVNEEHLGNKGAIKCIFPVITNTLKQKKVDTKNIDNIFSKRNMVTNFNVFEKILDLSLKDS
RLTNKFLTFFENINEFGFSSLSASNIVKKYFSNNSKGLKEKIDHYRKNNENQELYQILLYMVVFEIDDLLNQEELLN
LIDLNIDDYSLILGTILYLKNSSYKLEKLLKKIDQLFINTHANYDVKTSRMAEKLWLFRYFFYFLNCKNIFSQKEIN
SYCQSQNYNSGQNGYQTELNWNYIKGQGKDLRANNFFNELIVKEVWLISCGENEDEKYLN
ORF7 (AbiK-like) [Staphylococcus aureus](24636606)
SEQ ID NO: 12
    1 MVNLKIRNLY NTILDSSFLI RYGYYDVRMK KIESKNINNE ELEDYYGKPD TFYLKIFGLQ
  61 DLYFLMKSED LRGFFNESDF KKNVDTEPIY FNTPKNNYVR REYKMPNVYS YLHLCFFIED
121 NKEEFINIFE NNVQSTSKYF NELNFNFKFT KKIEQRLLFG GNSILSLDLS NFYHTLYTHS
181 IPWVIHGKQN SKDNRYKGFA NNLDSLIQKC QYGETHGIPV GNIISRIIAE LYMCYIDKKL
241 IEKGYKYARY VDDIKYPFVS NTDKEGFLME FNSICREYNL ILNDKKTDVQ TFPYRNNMQK
301 VEIFSYLDSL NKSSEIADWK SKINDFIDFC LSEEVSGNKG AVKCIYSVVI NKLRDSKMSS
361 NKINNILVSR EKLTKYNLYE KFLDISLKDS RLTNKFINFT EQLIDLKISK EKLKKIARQY
421 FKENKEIWKG NLDYYILNGW NQEVYQILLY SVLFDEEKIL NKKSLQLILK NDLDDYSKCL
481 SVILWIKKKF SFKVLLNTLE DKLKEVHSSY NDDASVRMQE KYWLLRYFIF YINKNNIIDD
541 AFFKKHYNEN NIKKDRNNII KSELNMHYVL KSDSRKKQYV NRVNEFFGFL LNNNVALIQT
601 NYNHKLFEYL
SEQ ID NO: 13
(https://www.ncbi.nlm.nih.gov/nuccore/AB057421.1?report = fasta)
atggtgaatttaaaaattcgtaatttatataacacgattttagatagttcttttctaattaggtatggatactatga
tgttagaatgaaaaagattgaatcaaaaaatattaataatgaagaattagaagattattatggcaaaccagatactt
tctatttaaaaatttttggtttgcaagacttatattttctgatgaaatcagaggatttaagaggattttttaatttc
tctgattttaaaaagaatgtagatacggaaccaatttattttaatactcctaaaaataattatgttcgtagagaata
taagatgccaaatgtctatagttacttacatctatgtttttttatagaagataataaagaggaatttatcaatattt
tcgaaaacaatgtacaatcaacttcaaaatattttaatgaattaaactttaattttaaattcactaagaaaatagag
cagaggcttctttttggagggaatagtattttgtctttagatttatcaaacttttatcatactttgtacacacatag
tataccttgggtcatacacggaaaacagaactcgaaagataatagatataaagggtttgctaataatttggatagct
taattcaaaaatgtcaatatggagaaactcatggtatacccgtggggaatattatatcaaggataatagcagaacta
tatatgtgctatatagacaaaaaattaattgaaaaaggatataaatatgctagatatgtagatgatattaaatatcc
atttgtatcaaatacagataaagaaggatttttgatggaatttaattccatatgtcgagagtacaatttaattctaa
atgataaaaaaacagatgtgcagacttttccatatcgtaataacatgcaaaaagtagagatttttagttatctagat
agcttgaataaaagctcggaaattgcggattggaaatcaaaaattaatgattttattgatttctgcttaagtgaaga
agtttcaggtaataaaggagcagtaaaatgcatttattcggtggtaataaataaacttagagattcaaaaatgtcct
caaataagataaacaatattcttgttagtagggaaaaattaacaaaatataacctatatgagaaatttttggatatt
tcattgaaagattcgaggttaacaaataaatttataaactttacagagcaattgatagacttaaaaattagtaaaga
aaaactaaagaaaatagcaaggcaatattttaaagaaaacaaggaaatatggaaaggaaatttagattattatattt
taaatggttggaatcaagaagtataccaaatattattatactctgttttattcgatgaagaaaaaatactaaatana
aaatctttacaattaatattaaaaaatgatttagatgattactcaaaatgtttgagtgtcattttgtggattaaaaa
gaaatttagctttaaagtgttgttaaatacattggaagataaattaaaagaagtgcattcttcttacaacgatgang
caagcgttagaatgcaagaaaaatattggttattaaggtactttattttttatataaataaaaataatattattgac
gatgcatttttcaaaaaacattacaatgaaaataatataaaaaaagataggaacaatatcataaagtctgaacttaa
tatgcactatgttttaaaaagtgattcgagaaaaaagcaatatgtaaacagagtgaatgagttttttggatttttat
taaataataatgtggcattaattcagacaaactataaccataaattatttgaatatctataa
Additional AbiK-like proteins:
>WP_253019558.1 MULTISPECIES: RNA-directed DNA polymerase (Lactococcus lactis)
SEQ ID NO: 22
MKKEFTELYDFIFDPIFLVRYGYYDISIPNKKMNTEKVEIDNDYGKSDSFYFKVENMESLANYLRSHDLKKHENGNKPLS
TEPVYFNIPKNIEARRQYKMPNLYSYMALNYYICDNKKEFIDVEMDNKFSTSKFFNQLSFDYPKTQEIRQTLLYGGIKKL
HLDLSNFYHTLYTHSIPWMIDGKSTAKKNRKKGFSNRLDTLITACQYEETHGIPTGNLLSRIITELYMCHFDKQMERKNF
VYSRYVDDFIFPFTLENEKQEFLNEFNLICRENNLLINDNKTKVDNFPFVDQSSKSDIFSFFENITSMNSNDKWIKEISD
FIDYCVNEEHLGNKGAIKCIFPVIKNTLKQKKVDTKNIDIIFSKRNMVTNFNVFEKILDLSLKDSKLINKELTFFENINE
FGFSSLSASNIVKKYFSNNSKGIKNKIDHYRKNNENQELYQILLYAVVFEIDDLLNQEELLNLIDSNIDDYSLILGTILY
LKNSSYKLDKLLKKIDSLFINTHANYNVNTSRMAEKLWLFRYFFYFLNCKDIISKIEINSYCKSKKYKSGPNGYQTELNW
NYIKGQGNDLRANDFFNELILAEVWLIYCGENEDFKYLN
>WP_256969077.1 RNA-directed DNA polymerase (Enterococcus faecalis)
SEQ ID NO: 23
MKKEFTDMKKEFTELYDFIFDPIFLVRYGYYDISIPNKKMNTEKVEIDNDYGKSDSFYFKVENMESLANYLRSHDLKKHF
NGNKPLSTEPVYFNIPKNIEARRQYKMPNLYSYMALNYYICDNKKEFIDVFMDNKESTSKFFNQLSFNYPKTQEIRQTLL
YGGIKKLHLDLSNFYHTLYTHSIPWMIDGKSTAKKNRKKGFSNRLDTLITACQYEETHGIPTGNLLSRIITELYMCHFDK
QMERKNFVYSRYVDDFIFPFTLENEKQEFLNEFNLICRENNLLINDNKTKVDNFPFVDQSSKSDIFSFFENITSMNSNDK
WIKEISDFIDYCVNEEHLGNKGAIKCIFPVIKNTLKQKKVDTKNIDIIFSKRNMVTNFNVFEKILDLSLKDSKLINKELT
FFENINEFGFSSLSASNIVKKYFSNNSKGIKNKIDHYRKNNENQELYQILLYAVVFEIDDLLNQEELLNLIDSNIDDYSL
ILGTILYLKNSSYKLDKLLKKIDSLFINTHANYNVNTSRMAEKLWLFRYFFYFLNCKDIISKIEINSYCKSKKYKSGPNG
YQTELNWNYIKGQGNDLRANDFFNELILAEVWLIYCGENEDFKYLN
>WP_227259543.1 RNA-directed DNA polymerase (Vagococcus fluvialis)
SEQ ID NO: 24
MKKEFTELYDFIFDPIFLVRYGYYDISIPNKKMNTEKVEIDNDYGKSDSFYFKVENMESLANYLRSHDLKKHENGNKPLS
TEPVYFNIPKNIEARRQYKMPNLYSYMALNYYICDNKKEFIDVFMDNKFSTSKFFNQLSFDYPKTQEIRQTLLYGGIKKL
HLDLSNFYHTLYTHSIPWMIDGKSTAKKNRKKGFSNRLDTLITACQYEETHGIPTGNLLSRIITELYMCHFDKQMERKNF
VYSRYVDDFIFPFTLENEKQEFLNEFNLICRENNLLINDNKTKVDNFPFVDQSSKSDIFSFFENITSMNSNDKWIKEISD
FIDYCVNEEHLGNKGAIKCIFPVIKNTLKQKKVDTKNIDIIFSKRNMVTNFNVFEKILDLSLKDSKLINKELTFFENINE
FGFSSLSASNIVKKYFSNNSKGIKNKIDHYRKNNENQELYQILLYAVVFEIDDLLNQEELLNLIDSNIDDYSLILGTILY
LKNSSYKLDKLLKKIDSLFINTHANYNVNNSRMAEKLWLFRYFFYFLNCKDIISKIEINSYCKSKKYKSGPNGYQTELNW
NYIKGQGNDLRANDFFNELILAEVWLIYCGDNEDFKYLN
>WP_081041261.1 RNA-directed DNA polymerase (Lactococcus lactis)
SEQ ID NO: 25
MESVFTELYDLMFDPVFLVRYGYYDITIKNKKMNTEKVEIENDYGKSDSFYFKVENMESFSEYLRGHDLKKFFKYGKPIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKFLTFFENMSS
LGFSSISASDIVKKYFSVNSNSIGRKIDYYHKNHFNQELYQILLYAVEFEIDNLLTQEELLKLINSNTDDESLVLVTILY
LKNSSYKRNELLEKIDSLFIDTHVNYPSDTARMSEKFWLFRYFFYFLQSKENINKKEVNTYCKSKNYNIGKNGYESELNW
RYIRGSASNTSVNDFFNELIENEVWLIYCGENGDFKYLPR
>WP_259683536.1 RNA-directed DNA polymerase (Lactococcus cremoris)
SEQ ID NO: 26
MESVFTELYDLIFDPVFLVRYGYYDITIKNKKMNTEKVEIENDYGKSDSFYFKVENMESFSEYLRGHDLKNFFKYEKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMMTGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASDIVKKYFSVNSKSIGRKIDYYHKNHFNQELYQILLYAVEFEIDNLLTQEELLKLITTNTDDFSLVLVTILY
LKNGSYKRNELLEKIDSLFIDTHVNYPSDTARMSEKFWLFRYFFYFLQSKENINKKEVNTYCKSKNYNIGKNGYESELNW
RYIRGSASNTSVNDFFNELIENEVWLIYCGENGDFKYLPR
>WP_270321368.1 RNA-directed DNA polymerase (Lactococcus petaurid)
SEQ ID NO: 27
MESVFTELYDLIFDPVFLVRYGYYDITIKNKKMNTEKVEIENDYGKSDSFYFKVENMESFSEYLRGHDLKKFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMMTGKAEAKKDRKNGFANTLDKLITSCQYDETHGIPTGNLLSRIIAELYMCHEDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASDIVKKYFSVNSKSIGRKIDYYHKNHFNQELYQILLYAVEFEIDNLLTQEELLKLIATNTDDFSLVLVTILY
LKNGSYKRNELLEKIDSLFIDTHVNYPSDTARMSEKFWLFRYFFYFLQSKENINKKEVNTYCKSKNYNIGKNGYESELNW
RYIRGSASNTSVNDFFNELIENEVWLIYCGENGDFKYLPR
>MDY5176804.1 RNA-directed DNA polymerase (Lactococcus lactis)
SEQ ID NO: 28
MESVFTELYDLIFDPVFLVRYGYYDITIKNKKMNTEKVEIENDYGKSDSFYFKVENMESFSEYLRGHDLKNFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMMTGKAEAKKDRKNGFANTLDKLITSCQYDETHGIPTGNLLSRIIAELYMCHEDKRMENNNF
IYTRYVDDIVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASDIVKKYFSVNSKSIGRKIDYYHKNHENQELYQILLYAVEFEIDNLLTQEELLKLITSNTDDFSLVLVTILY
LKNGSYKRNELLEKIDSLFIDTHVNYPSDTARMSEKFWLFRYFFYFLQSKENINKKEVNTYCKSKNYNIGKNGYESELNW
RYIRGSASNTSVNDFFNELIENEVWLIYCGGNGDFKYLPR
>WP_165719325.1 RNA-directed DNA polymerase (Lactococcus petaurid)
SEQ ID NO: 29
MESVFTELYDLIFDPVFLVRYGYYDITIKNKKMNTEKVEIENDYGKSDSFYFKVENMESFSEYLRGHDLKKFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASEIVKKYFSVNSNSIGRKIDYYHKNHFNQELYQILLYAVEFEIDNLLTQEELLKLITSNTDDESLVLVTILY
LKNGSYKRNELLEKIDSLFIDTHVNYPSDTARMSEKFWLFRYFFYFLLSKENINKKEVNTYCKSKNYNIGKNGYESELNW
RYIRGSASNTSVNDFFNELIENEVWLIYCGENGDFKYLPR
>WP_081199340.1 RNA-directed DNA polymerase (Lactococcus lactis)
SEQ ID NO: 30
MESVFTELYDLIFDPVFLVRYGYYDITIKNKKMNTEKVEIENDYGKSDSFYFKVENMESFSEYLRGHDLKNFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASEIVKKYFSVNSNSIGRKIDYYHKNHFNQELYQILLYAVEFEIDNLLTQEELLKLITSNTDDESLVLVTILY
LKNGSYKRNELLEKIDSLFIDTHVNYPSDTARMSEKFWLFRYFFYFLLSKENINKKEVNTYCKSKNYNIGKNGYESELNW
RYIRGSASNTSVNDFFNELIENEVWLIYCGENGDFKYLPR
>WP_259751088.1 RNA-directed DNA polymerase (Lactococcus cremoris)
SEQ ID NO: 31
MESVFTELYDLIFDPVFLVRYGYYDITIKNKKMNTEKVEIENDYGKSDSFYFKVENMESFSEYLRGHDLKNFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMMTGKAEAKKDRKNGFANTLDKLITSCQYDETHGIPTGNLLSRIIAELYMCHEDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITNFNIFEKILDLSLKDSRLTNKELTFFENMSS
LGFSSISASEIVKKYFSANSNSIGRKIDYYHKNHFNQELYQILLYAVEFEIDNLLTQEELLKLITSNTDDESLVLVTILY
LKNGSYKRNELLEKIDSLFIDTHVNYPSDTARMSEKFWLFRYFFYFLLSKENINKKEVNTYCKSKNYNIGNNGYESELNW
RYIRGSASITSVNDFFNELIENEVWLIYCGENGDFKYLPR
>WP_270695689.1 RNA-directed DNA polymerase (Lactococcus cremoris)
SEQ ID NO: 32
MESVFTELYDLIFDPVFLVRYGYYDITIKNKKMNTEKVEIENDYGKSDSFYFKVENMESFSEYLRGHDLKKFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMMTGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITNFNIFEKILDLSLKDSRLTNKELTFFENMSS
LGFSSISASEIVKKYFSANSNSIGRKIDYYHKNHFNQELYQILLYAVEFEIDNLLTQEELLKLITSNTDDFSLVLVTILY
LKNGSYKRNELLEKIDSLFIDTHVNYPSDTARMSEKFWLFRYFFYFLLSKENINKKEVNTYCKSKNYNIGNNGYESELNW
RYIRGSASITSVNDFFNELIENEVWLIYCGENGDFKYLPR
>WP_311792723.1 RNA-directed DNA polymerase (Lactococcus petaurid)
SEQ ID NO: 33
MESVFTELYDLIFDPVFLVRYGYYDITIKNKKMNTEKVEIENDYGKSDSFYFKVENMESFSEYLRGHDLKKFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCYFDKRMENNNF
IYTRYVDDVVFPFTLEAEKEDFLKEFSLICRENNLLVNDNKTRVNNFPFTNKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKFLTFFENMSS
LGFSSISASEIVKKYFSVNSSSIGRKIDYYHKNHFNQELYQILLYAVEFEIDNLLTQEELLKLITSNTDDFSLVLVTILY
LKNGSYKRNELLEKIDSLFIDTHVNYPSDTARMSEKFWLFRYFFYFLQSKENINKKEVNTYCKSKNYNIGKNGYKSELNW
RYIRGSASNTSVNDFFNELIENEVWLIYCGENGDFKYLPR
>WP_270502917.1 RNA-directed DNA polymerase, partial (Lactococcus petaurid)
SEQ ID NO: 34
KVEIENDYGKSDSFYFKVENMESFSEYLRGHDLKKFFKYGKSISTEPVYFSIPKNINSRRQYKMPNLYSYMALNYYMCDQ
KKEFVDVFVSNKFSTSKFFNQLNFDYSTTQEISQTLLYGGVKKLYLDLSNFYHTLYTHSIPWMITGKAEAKKDRKNGFAN
TLDKLITSCQYDETHGIPTGNLLSRIIAELYMCHFDKRMENNNFIYTRYVDDVVFPFTLEAEKEDFLKEFSLICRENNLL
VNDNKTRVDNFPFINKSSKSNIFSFFENLTLKNSDEKWIKEISNFIDYCINEESLGNKGAIKSIFPVIKNTFKNKKISSA
KLNNIFSKKDIITDFNIFEKILDLSLKDSRLTNKELTFFENMSSLGFSSISASEIVKKYFSVNSNSIGRKIDYYHKNHEN
QELYQILLYAVEFEIDNLLTQEELLKLITSNTDDFSLVLVTILYLKNGSYKRNELLEKIDSLFIDTHVNYPSDTARMSEK
FWLFRYFFYFLLSKENINKKEVNTYCKSKNYNIGKNGYESELNWRYIRGSASNTSVNDFFNELIENEVWLIYCGENGDFK
YLPR
>WP_270228270.1 RNA-directed DNA polymerase, partial (Lactococcus garvieae)
SEQ ID NO: 5
FYFKVENMESFSEYLRGHDLKKFFKYGKSISTEPVYFSIPKNINNRRQYKMPNLYSYMALNYYMCDQKKEFVDVFVSNKF
STSKFFNQLNFDYSTTQEISQTLLYGGVKKLYLDLSNFYHTLYTHSIPWMITGKADAKKDRKNGFANTLDKLITSCQYDE
THGIPTGNLLSRIIAELYMCYFDKRMENNNFIYTRYVDDVVFPFTLEAEKEDFLKEFSLICRENNLLVNDNKTRVDNFPF
INKSSKSNIFSFFENLTLKNSDEKWIKEISNFIDYCINEESLGNKGAIKSIFPVIKNTFKNKKISSAKLNNIFSKKDIIT
DFNIFEKILDLSLKDSRLTNKFLTFFENMSSLGFSSISASEIVKKYFSVNSSSIGRKIDYYHKNHFNQELYQILLYAVEF
EIDNLLTQEELLKLITSNTDDFSLVLVTILYLKNGSYKRNELLEKIDSLFIDTHVNYPSDTARMSEKFWLFRYFFYFLQS
KENINKKEVNTYCKSKNYNIGKNGYESELNWRYIRGSASNTSVNDFFNELIENEVWLIYCGENGDFKYLPR
>WP_300697012.1 RNA-directed DNA polymerase (uncultured Clostridium sp.)
SEQ ID NO: 36
MESVFTELYDLMEDPVFLVRYGYYDITIKNKKMNTEKVEIENDYGKSDSFYFKVENMESFSEYLRGHDLKKFFKYGKPIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASDIVKKYFSVNSNSIGRKIDYYHKNHFNQELYQILLYAVEFEIDNLLTQEELLKLINSNTDDFSLVLVTILY
LKNSSYKRNELLEKIDSLFIDTHVNYPSDTARMSEKFWLFRYFFYFLHK
>WP_285118722.1 RNA-directed DNA polymerase, partial (Lactococcus petaurid)
SEQ ID NO: 37
MESVFTELYDLIFDPVFLVRYGYYDITIKNKKMNTEKVEIENDYGKSDSFYFKVENMESFSEYLRGHDLKKFFKYGKSIS
TEPVYFSIPKNINSRRQYKMPNLYSYMALNYYMCDQKKEFVDVFVSNKFSTSKFFNQLNFDYSTTQEISQTLLYGGVKKL
YLDLSNFYHTLYTHSIPWMITGKAEAKKDRKNGFANTLDKLITSCQYDETHGIPTGNLLSRIIAELYMCHFDKRMENNNF
IYTRYVDDVVFPFTLEAEKEDFLKEFSLICRENNLLVNDNKTRVDNFPFINKSSKSNIFSFFENLTLKNSDEKWIKEISN
FIDYCINEESLGNKGAIKSIFPVIKNTFKNKKISSAKLNNIFSKKDIITDFNIFEKILDLSLKDSRLTNKELTFFENMSS
LGFSSISASEIVKKYFSVNSNSIGRKIDYYHKNHFNQELYQILLYAVEFEIDNLLTQEELLKLITSNTDDFSLVLV
>OHY29199.1 hypothetical protein BI362_11970 (Streptococcus parauberis)
SEQ ID NO: 38
MCHFDKQMERKNFVYSRYVDDFIFPFTLENEKQEFLNEFNLICRENNLLINDNKTKVDNFPFVDQSSKSDIFSFFENITS
MNSNDKWIKEISDFIDYCVNEEHLGNKGAIKCIFPVIKNTLKQKKVDTKNIDIIFSKRNMVTNFNVFEKILDLSLKDSKL
TNKFLTFFENINEFGFSSLSASNIVKKYFSNNSKGIKNKIDHYRKNNENQELYQILLYAVVFEIDDLLNQEELLNLIDSN
IDDYSLILGTILYLKNSSYKLDKLLKKIDSLFINTHANYNVNTSRMAEKLWLFRYFFYFLNCKDIISKKEINSYCESKKY
KSGPNGYQTELNWNYIKGQGNDLRANDFFNELILAEVWLIYCGENEDFKYLN
>WP_205288036.1 RNA-directed DNA polymerase (Lactococcus cremoris)
SEQ ID NO: 39
MMTGKAEAKKDRKNGFANTLDKLITSCQYDETHGIPTGNLLSRIIAELYMCHEDKRMENNNFIYTRYVDDVVFPFTLEAE
KEDFLKEFSLICRENNLLVNDNKTRVDNFPFINKSSKSNIFSFFENLTLKNSDEKWIKEISNFIDYCINEESLGNKGAIK
SIFPVIKNTFKNKKISSAKLNNIFSKKDIITNFNIFEKILDLSLKDSRLTNKELTFFENMSSLGFSSISASEIVKKYFSA
NSNSIGRKIDYYHKNHFNQELYQILLYAVEFEIDNLLTQEELLKLITSNTDDFSLVLVTILYLKNGSYKRNELLEKIDSL
FIDTHVNYPSDTARMSEKFWLFRYFFYFLLSKENINKKEVNTYCKSKNYNIGNNGYESELNWRYIRGSASITSVNDFFNE
LIENEVWLIYCGENGDFKYLPR
Abi-P2
```

Abi-P2 proteins are encoded by P2-like prophages such as Enterobacteria phage P2-EC30 (orf570) and P2-EC58 (orf544)) (9). These genes and the proteins they code for are known to the art and are also available commercially (e.g., the synthetic gene that codes for Abi-P2 (residue 1-541) of E. coli prophage EC30 can be purchased from BioBasic and cloned into an expression vector to produce recombinant protein that can be purified for use in the methods provided herein).

```
SEQ ID NO: 3:
atgaaaaaagtatatgaactaaccagtgaagaagcactgtcatattttcttcgccatgactcctacacaaccttaga
attaccggcttatattaatttcaccacattattaaatgatattaattcatctatccataacaaaaaaattaaaattg
aaccaaccgccaaggagctgatgggtaaagatatcaattatgaggtgcttgtcagtaaagatggtctatatagctgg
cgtaggataacacttatcaatcccctttattatgtctacttctgtagaaaaatcacagcaccagcaacctgggaaat
cataacagaaaaattcaaatcttttgaatcaaacgacctttttacatgttcaagcatccccgtcagaaaagacaact
cgtcaaacattgctgcgtctgtaatgaattggtgggaagattttgaacaaaaaagccttgcccttgctcttgaatac
gaattcatgttcagcactgacatctcaaacttctacccatcaatatatactcatagttttgaatgggtattcatatc
aaaagaagaggcaaagaagaaaaaaagcaaaaataacccaggaggattaattgacagccacattcaaatgatgatga
acaaccagacaaatggtattccactcggcagcacattgatggatacatttgctgagcttatcttgggtcaaatcgat
atagaattaagaaaaaaaactaacgaactcaaaataataaactacaaggtagtacgctatcgtgatgattaccggat
cttctctaatagcaaagatgatttagacataatatcaaaatgtttagtcaatgtattgggcgattttggtttagatc
taaactcaaaaaaaactgaactatatgaagacatcatacttcattcgttgaaacaagctaaaaaagactacatcaaa
gaaaaaagacataagtcactccagaaaatgctcta
SEQ ID NO: 4:
MKKVYELTSEEALSYFLRHDSYTTLELPAYINFTTLLNDINSSIHNKKIKIEPTAKELMGKDINYEVLVSKDGLYSW
RRITLINPLYYVYFCRKITAPATWEIITEKFKSFESNDLFTCSSI PVRKDNSSNIAASVMNWWEDFEQKSLALALEY
EFMESTDISNEYPSIYTHSFEWVFISKEEAKKKKSKNNPGGLIDSHI QMMMNNQTNGI PLGSTLMDTFAELILGQID
IELRKKTNELKIINYKVVRYRDDYRIFSNSKDDLDIISKCLVNVLGDFGLDLNSKKTELYEDIILHSLKQAKKDYIK
EKRHKSLQKML
Abi - P2 Synechococcus sp (427996238)
SEQ ID NO: 5
    1 MPKYFSFKEL LEEIAKEVNG KKLSDYYGST IDSAGKQKLS RPCYYENVNH RFLNNKDGKF
  61 AWRPFQIIHP AIYVSLVNEI TSENNWREIV AAFKRFSENN QIKCLSIPIE SENLLSDKAE
121 NISNWWHSVE QNSIELSLKY EYIMHTDISD CYGSIYTHSI VWALHTKKIA KEQRRDRSLI
181 GNIIDSHIQD MSFAQTNGIP QGSGLMDLIA EIVLGFADLE LSEKLESYPH LIDYQILRYR
241 DDYRVFTNNP QESDLIVKSL TEILGNLGLK LNSQKTLSSN TVVTHSIKPD KLYWITNQIK
301 MEKLQDSLLF IHDLSCKYPN SGSLCRALDS FFDKISKVNK ERDDIIVMIS ILVDIMLKNP
361 RTYPIGAGIL SKLISLIKPI DKQVEILKTI IYRFQKLPNI GYLEIWIQRI ALKIDKEITE
421 GELLCKKLNN KNSNISLWNS DWIANNKLKK IINDQLIVNE KTVENMSKVV KGNEVKLFYS
481 KNNYYF
SEQ ID NO: 6
ttgccaaagtacttcagctttaaggaacttcttgaggaaattgccaaagaagttaatgggaagaaactatctgacta
ttatgggagtactatagactcagcaggaaaacaaaaactatcacgaccctgttactatgaaaatgttaaccatagat
ttctaaataataaagatggcaagtttgcttggagaccttttcaaataattcatccagctatctatgtatctcttgta
aatgaaattacatccgaaaataattggagagagattgttgctgcttttaagcgttttagctttaataatcagattaa
atgcttatctattccaattgaaagcgaaaatttactatcagataaggctgaaaatataagcaattggtggcattcag
ttgagcaaaattcgattgagctatcgcttaagtatgagtatattatgcatacggatatttctgattgttatggatca
atatacactcattcaatagtatgggcattacatacaaagaaaattgctaaagaacaaagaagagatcgttctctaat
tggaaatataattgactcacatatacaagatatgtcatttgctcaaacaaatggaattcctcaaggtagtgggttga
tggacttaatagctgaaattgttttgggctttgccgatcttgaattatcagaaaagttggaaagctatccacactta
atagattatcaaatcttaagatatagagatgattacagagtttttacaaataatccacaagaatctgacttaattgt
aaagtccttaactgagatacttggtaatttggggttaaaacttaattcacaaaaaacattatcttccaatacagttg
ttactcactctattaagccagacaaattatattggataacaaatcagataaagatggaaaaattacaagattcttta
ttatttatacatgatttatcctgtaaatatcctaactctggaagtctatgtagggcattagatagcttctttgacaa
aattagtaaagtcaataaagaaagagatgatattatagttatgataagtattcttgttgatattatgctcaaaaatc
caagaacttatcctattggggcaggaatactaagtaagttgatttctctgattaagccaatagataagcaggttgag
attcttaaaactataatttatagatttcaaaagttacctaatataggttatcttgaaatttggattcagagaattgc
tttaaaaatcgataaggaaataacttttggcgagttattatgtaagaaactaaataataaaaattcaaatatttcct
tatggaattcagattggatagcaaataataaattaaaaaaaattattaatgatcaactaattgtgaacgagaagact
gttgaaaatatgagtaaagtagttaaagggaatgaagtaaagcttttttattcaaaaaataattattatttctaa
Abi-P2 Oenococcus oeni (118587264)
SEQ ID NO: 7
    1 MEMGQRMTNG DARKRRNLLD MSNAEARDFF LKPESYITFE LPEYFSFDTV LTTASQSLED
  61 ASLASMTAKG KSLSNVADVN YKMLISKDGQ FDWRPFQIIH PVTYVDLANC ITEESNWKKI
121 IKRFEEFEAN PRIRCISIPV ESLTSQKDTA ATILNWWENL EQASIEYSLD YAYCIKTDIT
181 NCYGSIYTHS ISWALHGKSW SKQHRKPSNG VGNRIDNKIQ HLQFGQTNGI PQGSTLFDFV
241 AEMVLGYSDL MLSNRLNEKK ISNYQIIRFR DDYRIFSNSK SDCEQIAKEL SDVLADLNMH
301 FNSKKTMLTT DIISAAVKPD KSYWIKTSPL IWFKQGKNTY YRLSLQKHLM QIYEFSIKFP
361 NSGSLKRALK EFLDRVSSLQ KCPDDLEQLI SIMSVLLLKN PIGVPLGVAI LSRLFVFIDQ
421 KQVSSLLTQ
SEQ ID NO: 8
atggaaatgggtcaaagaatgactaacggtgatgcaagaaagagacgtaatttattagatatgtcgaacgctgaggc
aagagatttttttctgaagcccgaaagttacattacttttgaattaccagaatattttagctttgacacagtcctga
ccacggcaagtcaatctttggaagatgcttctctcgcaagcatgacggcgaagggcaaaagtctttctaatgtagct
gatgttaattataaaatgctgataagtaaagatggccaattcgattggcggccctttcaaatcattcatccagtgac
gtatgttgatttggctaactgcataacagaagagtcgaattggaagaaaataatcaaacgatttgaggaatttgaag
caaatcctagaattagatgtattagcattccggtggaatctcttacgagtcaaaaagatacagcagccacgatttta
aattggtgggaaaatttggaacaagcctcaatcgaatatagcttggattatgcctactgtatcaaaacagatattac
taattgctatggttcaatctatacacattcaatttcttgggcattgcatggcaagagctggtctaaacagcatagga
agccctcaaatggagttggtaaccgaattgacaacaagatacagcatttacaatttggacaaacgaatgggattcca
caagggagtacgctgtttgattttgtagcagaaatggttttgggatattccgatctgatgttatccaatcgtctcaa
tgagaaaaaaattagcaattatcagattattcgtttcagggacgattaccgaattttttctaattcgaaaagcgatt
gtgaacagattgctaaggaactctccgatgtgcttgctgatctcaatatgcattttaattcgaaaaaaacaatgttg
accactgatattatttcagcggcagttaaaccagataaaagttattggataaaaactagtcccttgatctggttcaa
acaaggcaaaaatacatactatcgcttaagtttgcagaaacatttaatgcagatttatgagttttcaataaaatttc
cgaattccggaagcttgaaaagggctctgaaggaattcttggacagagtatcaagcttacagaaatgtccggacgac
ttagagcaacttattagtattatgtctgttcttttattgaaaaatcctatcggcgttcctttgggcgttgctatatt
gagccggttatttgtgtttattgatcagaaacaagtttcttctttgttgacgcaatga
Putative reverse transcriptase [Enterobacteria phage P2-EC58](84619238)
SEQ ID NO: 9
    1 MHFLNCSHSH RYHDRKQVVI CAQLKKKSLI MKKIYELTTG RALKYFLQHD SYTTLELPSY
  61 VDFSSLLEEI NSAIDEGKIN FQPDSKSLMG KNINYEVLVS KDGLYSWRRI TLINPLYYVY
121 FCKLITSPSN WKAIRNKFRE FESNDLFLCS STPVSKKNTS NVAASVLNWW EDFEQKSLSL
181 ALEYEFMFST DISNFYPSIY THSFEWVFIS KEEAKKKENN NNPGRLIDTH IQMMMSNQTN
241 GIPLGSTLMD TFAELILGEI DLQLRKKTEE QKITDYKVVR YRDDYRIFSS SKDDLDKISK
301 CLVEVLGEFG LDLNSRKTEL HDDIILHSLK SAKKEYIIER SENSLQKMLY AIYLFSLKHQ
361 NSKITVRYLN DELRKLFRKK KITNSGHQLD AMLGIISSIM AKNPTTYPVG MAIFAKLLTF
421 LYDDDELKFG KLQQLHCKLG KQPNTEMLDI WFQRVQGKIH TQWEGDYKTA LCQRINDELK
481 EKKTFTIDGL WDVEWIPGSA KNKNKQKILS ILKKTKIVDL DAFEEMDNDI TPEEVNLEDK
541 EHSA
SEQ ID NO: 10
atgcacttcctgaattgctcgcattcgcatcgttatcatgatagaaaacaggtagttatttgtgcgcaacttaagaa
gaaatcactaattatgaaaaagatttatgaattaactactggtagagcgcttaagtattttctgcagcatgattcat
acactactctggagctacccagttatgtcgatttttcttccttgcttgaagaaatcaactccgcgatagatgaaggt
aaaatcaacttccaacctgactccaagtcattgatggggaagaatataaattacgaggttttagtcagcaaagatgg
cttatatagctggcgacgaataacactaattaatcctctgtactacgtgtatttttgtaaacttattacatcccctt
ccaactggaaggctataaggaataaatttagagagtttgagtctaatgatctttttttatgctcaagtaccccagtg
agcaaaaagaacacctcaaacgtagctgcatctgttttaaactggtgggaagattttgaacaaaaaagtctttcatt
ggctcttgagtatgagttcatgttcagcacagatatttcaaacttctacccttctatttacactcatagctttgaat
gggtattcatctcaaaagaagaggccaaaaagaaagaaaataacaataacccaggacgattgattgacactcatatc
caaatgatgatgagtaatcagacaaacggaataccattgggtagtacgttgatggatacatttgccgaattaatttt
aggcgaaattgatttacagctaagaaaaaaaactgaagagcaaaaaataacggattacaaagtagttcgctacagag
atgattatcgaatattttcaagcagtaaagatgatttggacaaaatctcaaagtgtttggttgaggtcttaggtgag
tttgggcttgatttaaactcaagaaaaacagaactacatgacgacatcattcttcactcccttaaatcagcaaaaaa
agaatatattatagaaaggtcgttcaactccctacagaaaatgttgtatgcaatatatttattttctttaaaacatc
aaaactccaaaattacagttagatatttaaatgatttcttgcgaaaattatttaggaagaaaaagatcacaaatagt
gggcatcaactagatgcaatgcttgggattatttcaagcatcatggctaaaaacccaaccacctacccagtgggaat
ggctattttcgcaaaactcttgaccttcctttatgacgacgatgaactcaaatttggcaagttacaacaacttcatt
gtaaactaggtaaacaaccaaatactgaaatgttagatatttggtttcaacgagttcaagggaagatacacacacaa
tgggaaggcgattacaaaacagccctatgtcaacgcataaatgatgaactcaaggaaaaaaaaacatttaccattga
tggcctgtgggatgtagagtggattccgggttcagccaaaaaaaaaacaaacagaaaatactatcaattttgaaaa
aaacaaaaattgtggatttagatgcattcgaagaaatggataatgatattacaccagaagaagtgaacttattcgac
aaggaacacagcgcttaa
Hypothetical protein LMHG_01920 [Listeria monocytogenes FSL N1-017](133728112)
SEQ ID NO: 11
    1 MIMRKKVDVL SMDNEKAREF FMKPSSYFSQ NLPKYFNLGY LLESAESKLG KKQLKKEMYF
  61 ENKVYSNFPN VNFLIQTNKT ISTYRPITLL HPYIYVDYVN FLTEIKIWEE LKERFQNLQE
121 EVKEKIICSS LPFDIETSKE DDSKKEMALN FWKSIEQETI KYSLGYNYLL KLDISNFYGS
181 IYTHTLCWAF HGENYSKEAK NSKNLQNLTG DKCDRKFQWM NYGETVGIPQ GNVISDLMSE
241 LLLAYIDSEL VKKIDDEIDY KIIRYRDDYR IFTKRLEDST LVKRELVVLL QRFKLNLGES
301 KTSQTTDIIS GSLKEDKMYW IEHDPVTKLT SDKFYRSPKI LMKKSLEEHK NKKYKTRVEN
361 EFFKKHFHNR TYQATLQKHL LIIKVFSDLY PNSGQLIAAL HEFEGRLLDL NYKDERNIGT
421 EVEVLIAILV DIIKKNPKIT EIGVKLLSTL LKKINFEEFE MKYLESNNSN EVQSDFEVKL
481 AYINSVNHKL SNSNYNDYLE IWMQRLVVKN LKESTTLSDL YIEQSKNELV KFCNSIIRGK
541 EMIQIFNEDW LKDEHKIELS KFINLNEIDK LTDIISDDEI YLAEYNQMT
```

In one embodiment, the Abi nucleic acid sequence can optionally code for, or the Abi protein sequence can optionally include, at least one tag to aid with purification, immobilization, and/or solubility. Examples of a tag can be His (HHHHHH (SEQ ID NO: 21), Myc (EQKLISEEDL; SEQ ID NO: 14), HA (e.g., YPYDVPDYA; SEQ ID NO: 15), GST (MSPILGYWKIKGLVQPTRLLLEYLEEKYEEHLYERDEGDKWRNKKFELGLEFPNLPYY IDGDVKLTQSMAIIRYIADKHNMLGGCPKERAEISMLEGAVLDIRYGVSRIAYSKDFETL KVDFLSKLPEMLKMFEDRLCHKTYLNGDHVTHPDFMLYDALDVVLYMDPMCLDAFP KLVCFKKRIEAIPQIDKYLKSSKYIAWPLQGWQATFGGGDHPPK; SEQ ID NO: 16), FLAG (e.g., DYKDDDD; SEQ ID NO: 17), CPB (KRRWKKNFIAVSAANRFKKISSSGAL; SEQ ID NO: 18), S-tag (KETAAAKFERQHMDS' SEQ ID NO: 19), or V5 (GKPIPNPLLGLDST; SEQ ID NO: 20), MBP (MKIEEGKLVIWINGDKGYNGLAEVGKKFEKDTGIKVTVEH PDKLEEKFPQVAATGDGPDIIFWAHDRFGGYAQSGLLAEITPDKAFQDKLYPFTWDAV RYNGKLIAYPIAVEALSLIYNKDLLPNPPKTWEEIPALDKELKAKGKSALMFNLQEPYFT WPLIAADGGYAFKYENGKYDIKDVGVDNAGAKAGLTFLVDLIKNKHMNADTDYSIAE AAFNKGETAMTINGPWAWSNIDTSKVNYGVTVLPTFKGQPSKPFVGVLSAGINAASPN KELAKEFLENYLLTDEGLEAVNKDKPLGAVALKSYEEELAKDPRIAATMENAQKGEI MPNIPQMSAFWYAVRTAVINAASGRQTVDEALKDAQTNSSSNNNNNNNNN NLGIEGR; SEQ ID NO: 40), or Tsf (MAEITASLVKELRERTGAGMMDCKKALTEANGDIELAIENMRKSGAIKAAKKAGNVA ADGVIKTKIDGNYGIILEVNCQTDFVAKDAGFQAFADKVLDAAVAGKITDVEVLKAQF EEERVALVAKIGENINIRRVAALEGDVLGSYQHGARIGVLVAAKGADEELVKHIAMHV AASKPEFIKPEDVSAEVVEKEYQVQLDIAMQSGKPKEIAEKMVEGRMKKFTGEVSLTG QPFVMEPSKTVGQLLKEHNAEVTGFIRFEVGEGIEKVETDFAAEVAAMSKQS; SEQ ID NO: 41). A tag may be fused at the N or C terminus of the protein or included internally.

In one embodiment, the Abi nucleic acid and/or protein have at least 70%, 75%, 80%, 85%, 90%, 95%, 96%, 97%, 98%, 99% or 100% sequence identity to any one of SEQ ID NOs: 1 to 13 and 22-39.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC5′ share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques like those described above, with or without allowing gaps. In calculating percent identity, typically exact matches are counted.

In another embodiment, the Abi protein has at least a TM (Template Modeling) value of about 0.5 when compared to any one of SEQ ID NOs: 2, 4, 5, 7, 9, 11 or 12.

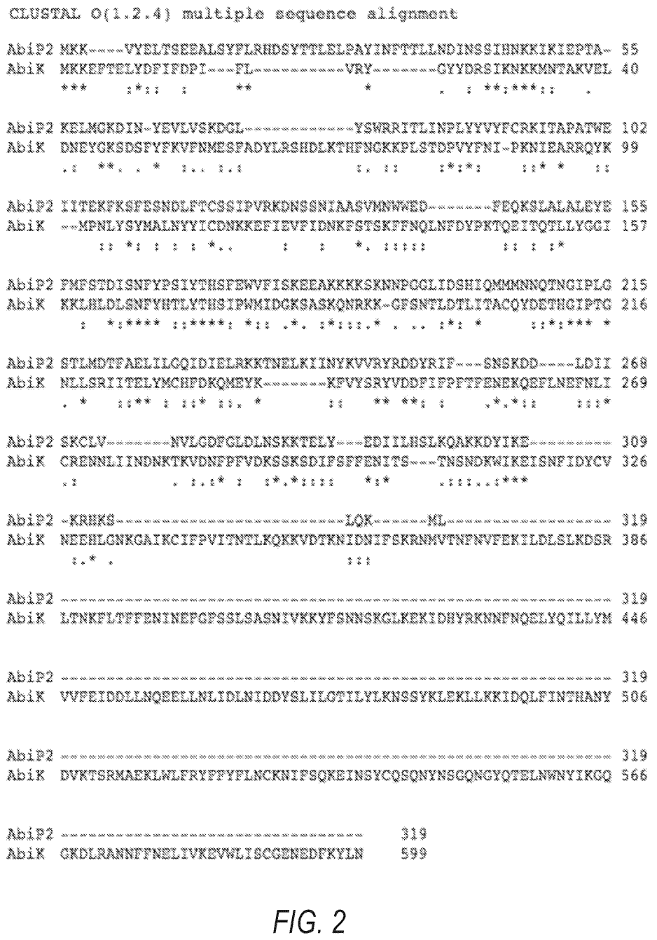

Proteins fold into complex tertiary structures that dictate their function. The amino acid sequence homology between AbiK and AbiP2 is only 32% as calculated by BLAST (FIG. 2) (https://blast.ncbi.nlm.nih.gov/Blast.cgi?PROGRAM=blastp&PAGE_TYPE=BlastSearch&BLA ST_SPEC=blast2seq&LINK_LOC=blasttab&LAST_PAGE=blastp&BLAST_INIT=blast2seq). Proteins are generally defined as ‘similar’ if they share 70% or greater percent similarity/identity. An overview of the sequencing alignment can be seen below in FIG. 2.

However, despite being divergent, protein sequences can still fold into very similar structure and preform similar functions (Pearson. Curr Protec Bioinformatics. 2013. “An Introduction to Sequence Similarity (“Homology”) Searching”). To evaluate protein structure similarity while also accounting for differences in protein length a different measurement can also used: Tm (template modeling) (Zhand and Skolnik. Nucleic Acids Res. 2005; 33(7): 2302-2309 (“TM-align: a protein structure alignment algorithm based on the TM-score”)). TM-align is described by Zhand and Skolnik and the TM align program is available at http://bioinformatics.buffalo.edu/TM-align and https://zhanggroup.org/TM-align.

Tm values range between 0 and 1. A Tm of one indicates a perfect match, and a Tm of 0.2 is defined as any two randomly chosen, unrelated structures being compared. A Tm value greater than or equal to about 0.5 is statistically likely to be related. Tm is commonly used in structural biology to determine relationships between proteins based on structure, as recently cited by AlphaFold, and is used in an international protein prediction competition (Critical Assessment of Techniques for Protein Structure Prediction—CASP) ((Jumper et al. Nature. 596, 583-589 (2021) “Highly accurate protein structure prediction with AlphaFold”) and (https://predictioncenter.org/casp14/results.cgi?view=tb-sel)).

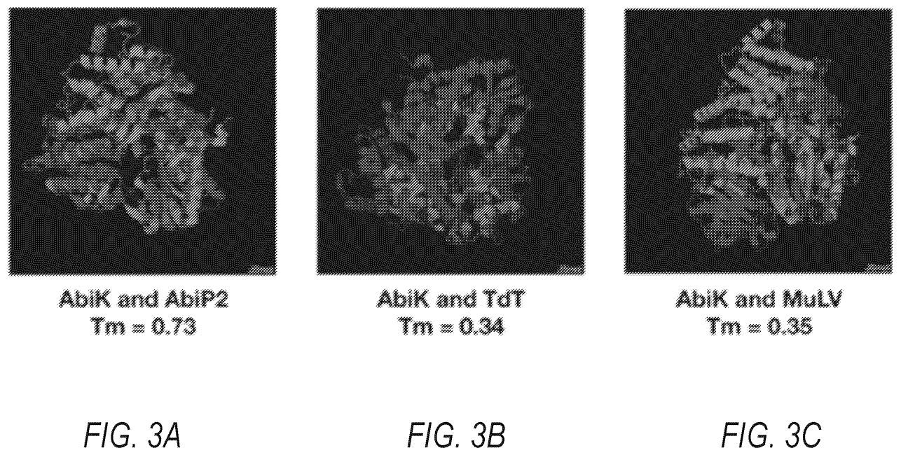

The sequence similarity between the AbiK and AbiP2 amino acid sequence is only 32% (FIG. 2). The Tm value when comparing the AbiK (PDB: 7R07) and AbiP2 (PDB: 7R08) structure is calculated to be 0.73 (FIG. 3) (https://zhanggroup.org/TM-align/tmp/148448.html). This is contrasted with an evaluation of the Tm value of AbiK compared to Terminal Deoxynucleotidyl Transferase (TdT) (PDB: 1K3J), another protein used in DNA enzymatic synthesis, in which the Tm calculated is 0.34, or Murine Leukemia Virus, another reverse transcriptase, (PDB: 4MH8) in which the Tm is 0.35.

As it is clear that AbiK and AbiP2 share a similar biological role in phage defense and have a common mechanism of untemplated DNA synthesis, not only is sequence homology able to express their relationship, but so is template modeling value. Evaluation of a structural score provides determination of protein family relationships. Therefore, a value of Tm≥0.5, including about 0.5, about 0.55, about 0.6, about 0.65, about 0.7, about 0.75, about 0.8, about 0.85, about 0.9, about 0.95, and about 1 can be used to determining structural similarity/identity between two proteins.

### Nucleotides/Nucleic Acids

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e., analogs having other than a phosphodiester backbone. For example, peptide nucleic acid (PNA), morpholino and locked nucleic acid (LNA), as well as glycol nucleic acid (GNA), threose nucleic acid (TNA) and hexitol nucleic acids (HNA). Each of these is distinguished from naturally occurring DNA or RNA by changes to the backbone of the molecule.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages.

The term nucleic acid also specifically includes nucleic acids composed of bases other than the five biologically occurring bases (adenine, guanine, thymine, cytosine, and uracil), including, but not limited to, non-naturally occurring nucleotides/nucleotide analogs such as 5-bromouracil (5BU), fluorescent based analogs (2-aminopurine (2-AP); 3-MI; 6-MI; 6-MAP; pyrrolo-dC; furan-modified bases, d5SICS, dNaM, 2-amino-8-(2-thienyl)purine (s); pyridine-2-one (y); 7-(2-thienyl)imidazo[4,5-b]pyridine (Ds); pyrrole-2-carbaldehyde (Pa); 4-[3-(6-aminohexanamido)-1-propynyl]-2-nitropyrrole (Px); xanthine; 5-(2,4 diaminopyrimidine) and many others).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

### Reversible Chain Terminating Nucleotides

A reversible terminator is a modified nucleotide analog that can terminate extension reversibly, which is widely used in various sequencing techniques. The termination effect is derived from the blocking groups in the molecule, which can be chemically or photochemically removed, allowing further extension of the DNA molecule, thus “reversible.” Such reversible terminators can include 3′-O-blocked reversible terminators and/or 3′-unblocked reversible terminators. They also include any reversible terminator available to an art worker, such as those described in Chen et al. Genomics Proteomics Bioinformatics 11 (2013) 34-40 (incorporated herein by reference).

In some embodiments, the reversible chain terminator nucleotide is at least one of:

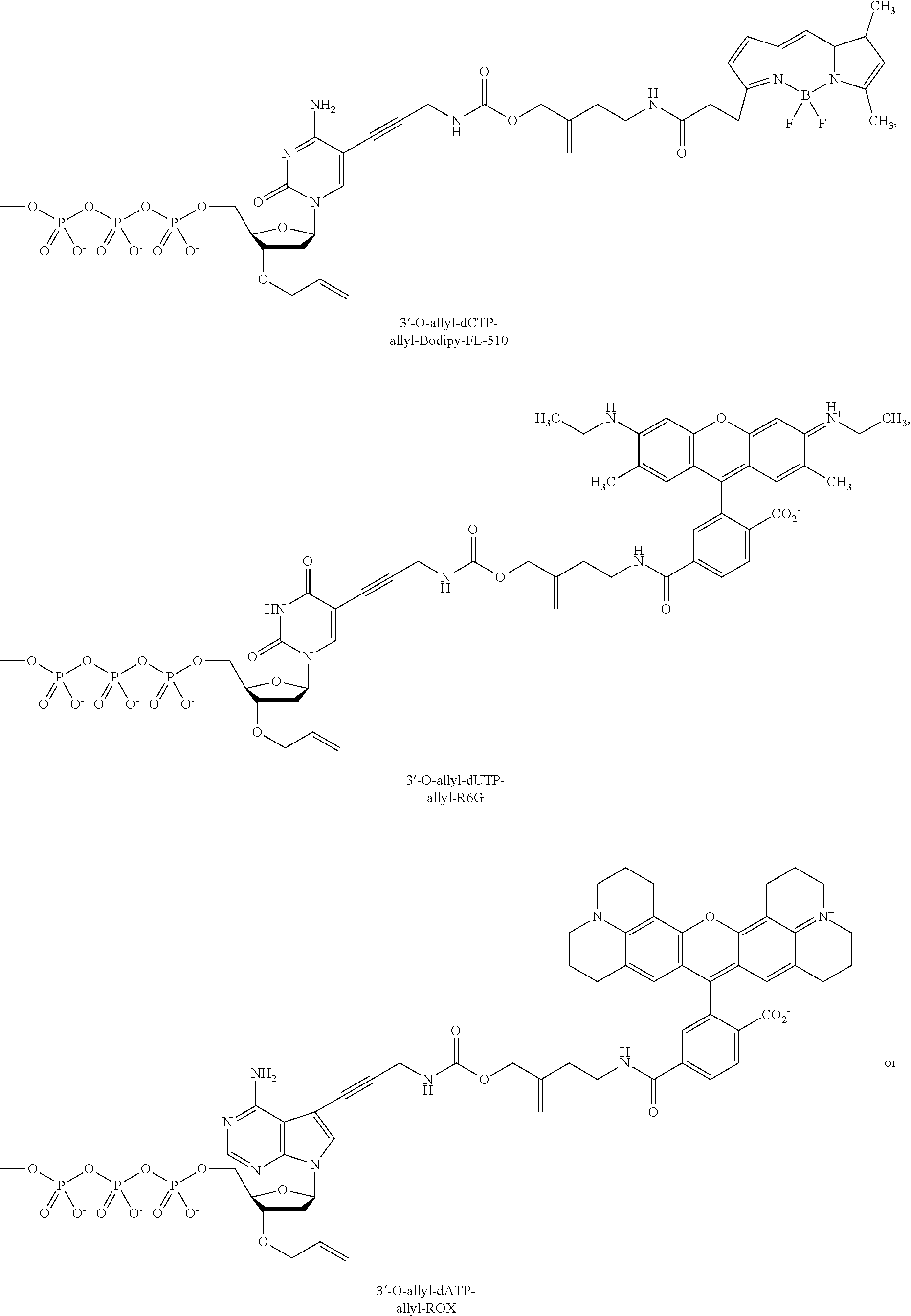

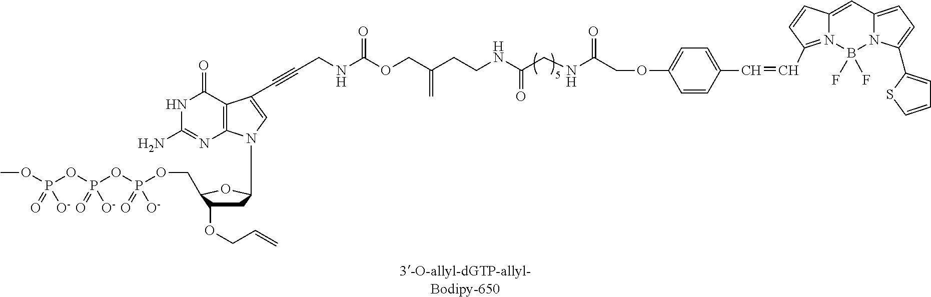

(as disclosed in Ju et al. 2006. Applied Biological Sciences. 103(52):19635-19640).

The reversible chain terminator nucleotides can be removed by specific wavelengths or appropriate chemical conditions.

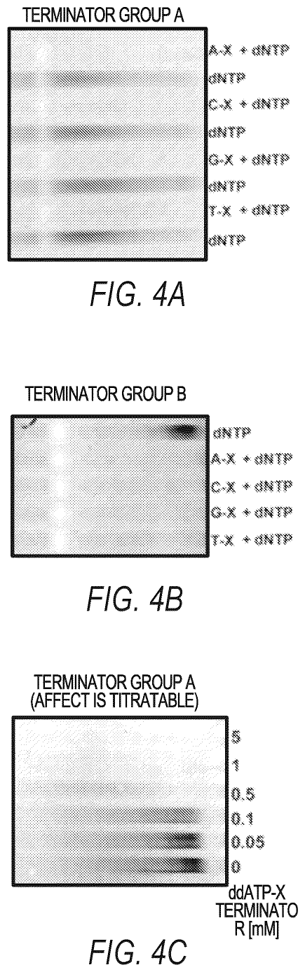

FIG. 4 demonstrates that AbiK can prevent the synthesis of DNA using two reversible terminators: Group A: 3′-O-Azidomehtyl-dCTP (Jena Biosciences) and Group B: 3″-aminoxy triphosphates (Firebird Biomolecular Sciences), indicating incorporation of the reversible terminator. For the data generated in FIG. 4, conditions for reversible terminator incorporation included 500 nM AbiK, 0.1 mM dNTP mix, 5 uM Fluorescein dUTP, 2 mM MgCl₂, 50 mM Tris pH8.0, 100 mM NaCl 5 mM DTT, 5 mM blocker. The reaction was incubated at 37° C. for 60 minutes. Following incubation with 2 μL of Proteinase K for another 15 minutes. Then 8 μL of reaction was added to 8 uL 2× Urea Loading Buffer and run on a 10% Urea loading gel. Gel was run for 45 minutes in 55C TBE loading buffer. Gel was then exposed for 2 minutes and imaged for fluorescein on a gel imager.

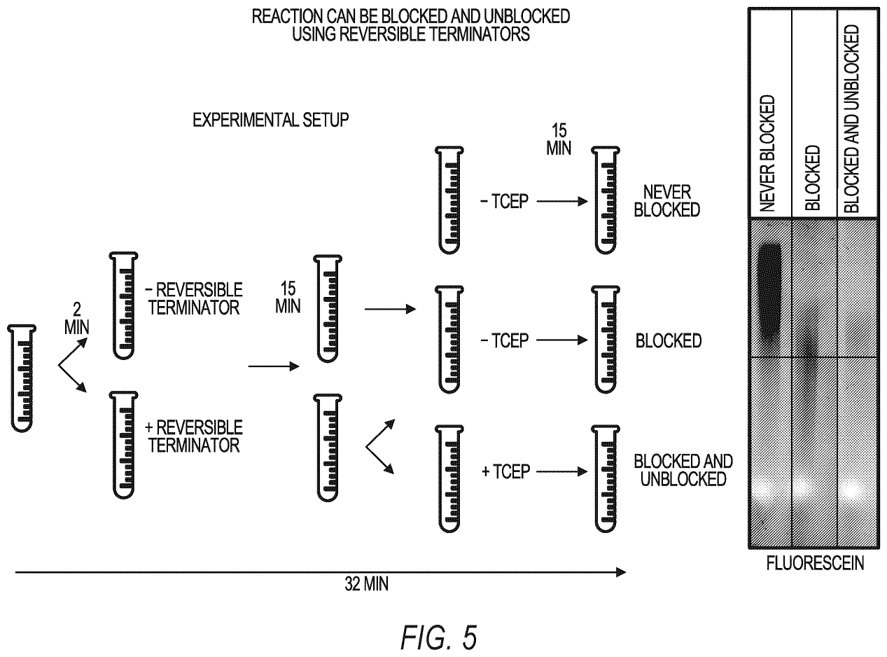

FIG. 5 demonstrates that the reaction can be blocked and unblocked using reversible terminators. The experimental setup as described in FIG. 4 except all components of the reaction were added, the reaction tube was allowed to sit at room temperature while the reaction ran. The reaction was performed at room temperature to ensure a sufficiently short DNA molecule would be synthesized to see a clear size difference on the gel. After two minutes ⅔ of the reaction by volume was split into another tube and reversible terminator Group A was added at a final concentration of 1 mM. Reactions were then incubated at 37° C. for 15 min. It is expected that during this time the reaction that was blocked would not expand in length and the reaction that was not blocked would expand in length. After 15 minutes, half of the blocked reaction had TCEP added to a concentration of 50 mM. TCEP should unblock the reversible terminator and if unblocking is successful the DNA being synthesized will continue to get longer. All reactions were allowed to run an additional 15 minutes at 37° C. Then the samples were treated with proteinase K to degrade the protein and run on a gel as described in FIG. 4.

### Solid Support

The Abi enzyme or the nucleic acid can be bound to a solid support, such as through a linker or tag that binds, for example, the protein to a surface. The solid support can be any substance that DNA or protein can be bound to, including but not limited to, paper, ceramic, gold, glass, metal, plastic, polystyrene, protein affinity tag resins, protein cleanup columns and/or silicone. One of skill in the art can bind the substances by methods available to an art worker.

### BIBLIOGRAPHY

1. Rostol, J. T. and Marraffini, L. (2019) (Ph)ighting phages: how bacteria resist their parasites. Cell Host Microbe, 25, 184-194.
2. Tock, M. R. and Dryden, D. T. (2005) The biology of restriction and anti-restriction. Curr. Opin. Microbiol., 8, 466-472.
3. Nussenzweig, P. M. and Marraffini, L. A. (2020) Molecular mechanisms of CRISPR-Cas immunity in bacteria. Annu. Rev. Genet., 54, 93-120.
4. Page, R. and Peti, W. (2016) Toxin-antitoxin systems in bacterial growth arrest and persistence. Nat. Chem. Biol., 12, 208-214.
5. Lopatina, A., Tal, N. and Sorek, R. (2020) Abortive infection: bacterial suicide as an antiviral immune strategy. Annu. Rev. Virol., 7, 371-384.
6. Emond, E., Holler, B. J., Boucher, I., Vandenbergh, P. A., Vedamuthu, E. R., Kondo, J. K. and Moineau, S. (1997) Phenotypic and genetic characterization of the bacteriophage abortive infection mechanism AbiK from Lactococcus lactis. Appl. Environ. Microbiol., 63, 1274-1283.
7. Odegrip, R., Nilsson, A. S. and Hagg°ard-Ljungquist, E. (2006) Identification of a gene encoding a functional reverse transcriptase within a highly variable locus in the P2-Like coliphages. J. Bacteriol., 188, 1643-1647.
8. Malgorzata Figiel, Marta Gapinska, Mariusz Czarnocki-Cieciura, Weronika Zajko, Malgorzata Sroka, Krzysztof Skowronek and Marcin Nowotny. (2022) Mechanism of protein-primed template-independent DNA synthesis by Abi polymerases. Nucleic Acids Research., 50(17), 10026-10040.

The embodiments are described in sufficient detail to enable those skilled in the art to practice the invention. Other embodiments may be utilized and formulation and method of using changes may be made without departing from the scope of the invention. The detailed description is not to be taken in a limiting sense, and the scope of the invention is defined only by the appended claims, along with the full scope of equivalents to which such claims are entitled.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

1. A method to synthesize a single strand DNA segment comprising
a) contacting an AbiK and/or Abi-P2 enzyme with a nucleotide, wherein the enzyme and the nucleotide form a complex;
b) removing any unbound nucleotide from a);
c) building the single strand DNA segment from the 3″ end of the nucleotide bound to the enzyme/nucleotide of b) by contacting the enzyme/nucleotide of b) with another nucleotide;
d) removing any unbound nucleotide from c); and
e) repeating the steps of contacting and removing free nucleotides until a desired length and sequence of the single strand DNA segment is formed.

2. The method of claim 1, wherein the enzyme is AbiK.

3. The method of claim 1, wherein the AbiK enzyme has the amino acid sequence provided in one of SEQ ID NO: 2, SEQ ID NO: 12, SEQ ID NO: 22-39 or 70% identity to SEQ ID NO: 2, SEQ ID NO: 12 or SEQ ID NO: 22-39 or a Template Molding (TM) value of at least about 0.5 when compared to SEQ ID NO: 2, SEQ ID NO: 12 or SEQ ID NO: 22-39.

4. The method of claim 1, wherein the enzyme is Abi-P2.

5. The method of claim 1, wherein the Abi-P2 enzyme has the amino acid sequence provided in SEQ ID NO: 4, SEQ ID NO: 5, SEQ ID NO: 7, SEQ ID NO:9, SEQ ID NO: 11 or 70% identity SEQ ID NO: 4, SEQ ID NO: 5, SEQ ID NO: 7, SEQ ID NO:9, or SEQ ID NO: 11 or a Template Molding (TM) value of at least about 0.5 when compared to one of SEQ ID NO: 4, SEQ ID NO: 5, SEQ ID NO: 7, SEQ ID NO:9 or SEQ ID NO:11.

6. The method of claim 1, wherein the nucleotides are bound to a reversible chain nucleotide terminator.

7. The method of claim 6, wherein the reversible chain nucleotide terminator comprises at least one 3′O-blocked reversible terminator and/or 3′-unblocked reversible terminator.

8. The method of claim 6, wherein the reversible chain nucleotide terminator comprises at least one of:

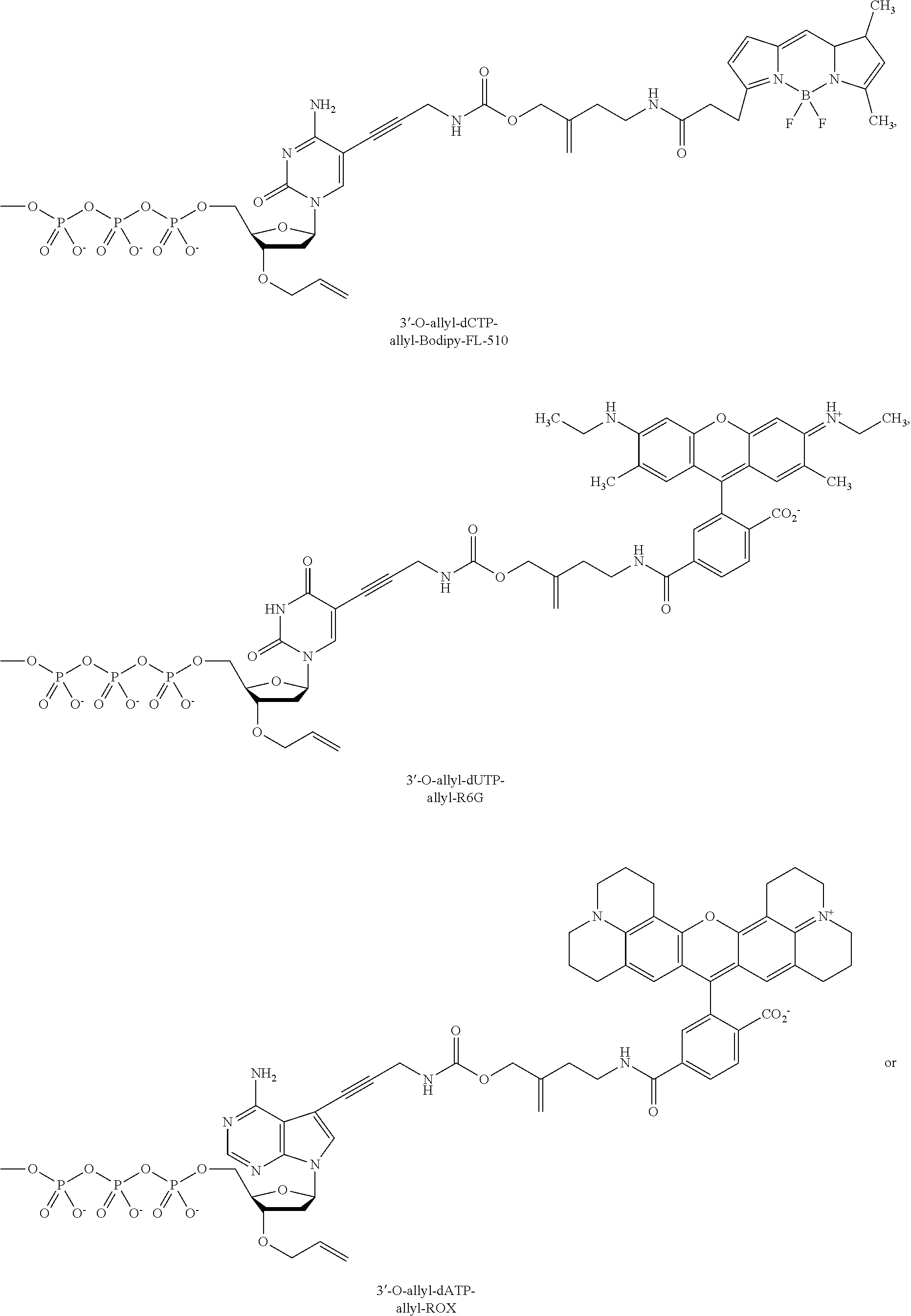

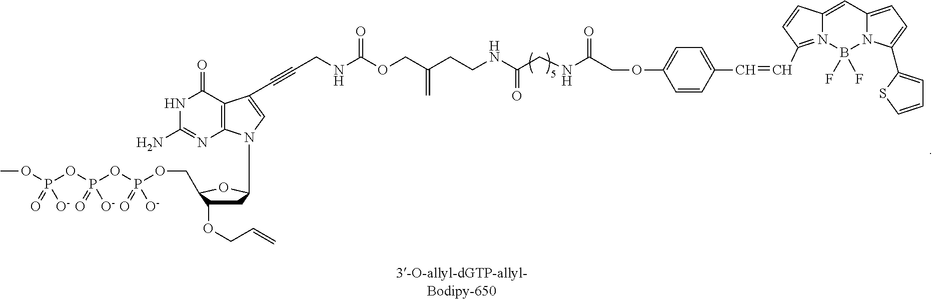

9. The method of claim 6, wherein the reversible chain nucleotide terminator is removed after removing any unbound nucleotide and prior to contacting with another nucleotide to build the single strand DNA segment.

10. The method of claim 1, wherein the nucleotides are independently selected from A, T, G, C or a nucleotide analog.

11. The method of claim 10, wherein the nucleotide analog comprises one or more non-naturally occurring nucleotides/nucleotide analogs including 5-bromouracil (5BUY), fluorescent based analogs (2-aminopurine (2-AP), 3-MI, 6-MI, 6-MA-P, pyrrolo-dC, furan-modified bases, d5SICS, dNaM, 2-amnion-8-(2-thienyl)purine (s), pyridine-2-one (v), 7-(2-thienyl)imidazo[4,5-b]pyridine (Ds), pyrrole-2-carbaldehyde (Pa), 4-[3-(6-aminohexanamido)-1-propynyl]-2-nitropyrrole (Px); xanthine, 5-(2,4 diaminopyrimidine) or a combination thereof.

12. The method of claim 1, wherein the single strand DNA segment is 1 to about 10,000 nucleotides long.

13. The method of claim 1, wherein the single strand DNA segment comprises naturally and non-naturally occurring nucleotides.

14. The method of claim 1, wherein the enzyme or nucleotide is attached to a solid substrate.

15. The method of claim 14, wherein the solid substrate is paper, ceramic, gold, glass, metal, plastic, polystyrene, protein affinity tag resin, protein cleanup column, silicone or a combination thereof.

16. A DNA synthesis device comprising:
a reaction chamber; and
the enzyme/nucleotide complex of claim 1, located at least partially within the reaction chamber.

17. The device of claim 16, wherein the reaction chamber is a well, a channel, a cartridge, a or a pore.

18. The device of claim 16, wherein the device is a flow cytometry device, a microarray or a 96 well plate.

19. The device of claim 16, wherein the device is an automated device.


SEQUENCE LISTING (41 sequences)

SEQ ID NO: 1 - DNA, 1800 bp, Lactococcus lactis
  source 1..1800: mol_type = other DNA
atgaaaaaagagtttactgaattatatgattttatatttgatcctatttttcttgtaagatacggctattatgatagatctattaaaaacaaaaaaatgaatactgcaaaagttgaattagacaatgaatatggaaaatcagattctttttattttaaagtatttaatatggaatcctttgcagattatttaaggagtcatgatttaaaaacacattttaacggtaaaaaacctctatcaacagacccagtatattttaatattccaaaaaatatagaagctagaagacaatataagatgcccaatttatacagttatatggcattaaattattatatatgtgacaataaaaaagagtttatagaagtatttattgataacaaattttcaacgtcaaaattttttaatcaattgaattttgattatcctaagacacaagaaattacacaaacattattatatggaggaataaagaaattacatttagatttatctaatttttatcatactttatatacacatagtataccatggatgattgatggaaaatctgcatctaaacaaaatagaaaaaaagggttttctaatacattagatactttgattacagcttgtcaatacgacgaaacacatggcattccaactggaaatctattgtctaggattattaccgaactatatatgtgccattttgataaacaaatggaatataagaagtttgtgtattcaagatatgtagatgattttatatttccgtttacttttgagaatgaaaagcaagaatttttaaatgaatttaatctaatctgtcgagaaaataacttaattattaatgataataaaacgaaagttgacaatttcccgtttgttgataaatcgagtaaatcggatattttttctttttttgaaaatattacttcaactaattccaacgacaagtggattaaagaaataagcaattttatagattattgtgtgaatgaagaacatttagggaataagggagctataaaatgtattttcccagttataacaaatacattgaaacaaaaaaaagtagatactaaaaatatagacaatatcttttcgaaaagaaacatggttaccaattttaatgttttcgaaaaaatattagatttatcattaaaagattcaagattaactaataagtttttgactttctttgaaaatattaatgaatttggattttcaagtttatcagcttcaaatattgtaaaaaaatattttagtaataattcaaagggcttaaaagaaaaaatagaccactatcgtaaaaataattttaatcaagaattatatcaaatattgttgtatatggttgtctttgaaatagatgatttattaaatcaagaagaattactaaacttaattgatttaaatattgatgattattctttaattttagggacgattttatacctaaagaatagttcatataaattggaaaaattattaaaaaaaatagatcaattatttattaatactcatgccaactacgacgttaaaacttctcgtatggcagaaaaattatggctatttcgttatttcttttattttttaaattgtaagaatatttttagtcaaaaagagataaatagttattgtcaatctcaaaactataattcaggacagaacggatatcaaacagaacttaattggaattatattaaaggtcaagggaaggatcttagagcgaataacttttttaatgaattgatagtaaaagaagtttggttaatttcttgtggtgagaacgaagatttcaaatatttaaattga

SEQ ID NO: 2 - protein, 599 aa, Lactococcus lactis
MKKEFTELYDFIFDPIFLVRYGYYDRSIKNKKMNTAKVELDNEYGKSDSFYFKVFNMESFADYLRSHDLKTHFNGKKPLSTDPVYFNIPKNIEARRQYKMPNLYSYMALNYYICDNKKEFIEVFIDNKFSTSKFFNQLNFDYPKTQEITQTLLYGGIKKLHLDLSNFYHTLYTHSIPWMIDGKSASKQNRKKGFSNTLDTLITACQYDETHGIPTGNLLSRIITELYMCHFDKQMEYKKFVYSRYVDDFIFPFTFENEKQEFLNEFNLICRENNLIINDNKTKVDNFPFVDKSSKSDIFSFFENITSTNSNDKWIKEISNFIDYCVNEEHLGNKGAIKCIFPVITNTLKQKKVDTKNIDNIFSKRNMVTNFNVFEKILDLSLKDSRLTNKFLTFFENINEFGFSSLSASNIVKKYFSNNSKGLKEKIDHYRKNNFNQELYQILLYMVVFEIDDLLNQEELLNLIDLNIDDYSLILGTILYLKNSSYKLEKLLKKIDQLFINTHANYDVKTSRMAEKLWLFRYFFYFLNCKNIFSQKEINSYCQSQNYNSGQNGYQTELNWNYIKGQGKDLRANNFFNELIVKEVWLISCGENEDFKYLN

SEQ ID NO: 3 - DNA, 959 bp, Escherichia coli
  source 1..959: mol_type = other DNA
atgaaaaaagtatatgaactaaccagtgaagaagcactgtcatattttcttcgccatgactcctacacaaccttagaattaccggcttatattaatttcaccacattattaaatgatattaattcatctatccataacaaaaaaattaaaattgaaccaaccgccaaggagctgatgggtaaagatatcaattatgaggtgcttgtcagtaaagatggtctatatagctggcgtaggataacacttatcaatcccctttattatgtctacttctgtagaaaaatcacagcaccagcaacctgggaaatcataacagaaaaattcaaatcttttgaatcaaacgacctttttacatgttcaagcatccccgtcagaaaagacaactcgtcaaacattgctgcgtctgtaatgaattggtgggaagattttgaacaaaaaagccttgcccttgctcttgaatacgaattcatgttcagcactgacatctcaaacttctacccatcaatatatactcatagttttgaatgggtattcatatcaaaagaagaggcaaagaagaaaaaaagcaaaaataacccaggaggattaattgacagccacattcaaatgatgatgaacaaccagacaaatggtattccactcggcagcacattgatggatacatttgctgagcttatcttgggtcaaatcgatatagaattaagaaaaaaaactaacgaactcaaaataataaactacaaggtagtacgctatcgtgatgattaccggatcttctctaatagcaaagatgatttagacataatatcaaaatgtttagtcaatgtattgggcgattttggtttagatctaaactcaaaaaaaactgaactatatgaagacatcatacttcattcgttgaaacaagctaaaaaagactacatcaaagaaaaaagacataagtcactccagaaaatgctcta

SEQ ID NO: 4 - protein, 319 aa, Escherichia coli
MKKVYELTSEEALSYFLRHDSYTTLELPAYINFTTLLNDINSSIHNKKIKIEPTAKELMGKDINYEVLVSKDGLYSWRRITLINPLYYVYFCRKITAPATWEIITEKFKSFESNDLFTCSSIPVRKDNSSNIAASVMNWWEDFEQKSLALALEYEFMFSTDISNFYPSIYTHSFEWVFISKEEAKKKKSKNNPGGLIDSHIQMMMNNQTNGIPLGSTLMDTFAELILGQIDIELRKKTNELKIINYKVVRYRDDYRIFSNSKDDLDIISKCLVNVLGDFGLDLNSKKTELYEDIILHSLKQAKKDYIKEKRHKSLQKML

SEQ ID NO: 5 - protein, 486 aa, Synechococcus aeruginosa
MPKYFSFKELLEEIAKEVNGKKLSDYYGSTIDSAGKQKLSRPCYYENVNHRFLNNKDGKFAWRPFQIIHPAIYVSLVNEITSENNWREIVAAFKRFSFNNQIKCLSIPIESENLLSDKAENISNWWHSVEQNSIELSLKYEYIMHTDISDCYGSIYTHSIVWALHTKKIAKEQRRDRSLIGNIIDSHIQDMSFAQTNGIPQGSGLMDLIAEIVLGFADLELSEKLESYPHLIDYQILRYRDDYRVFTNNPQESDLIVKSLTEILGNLGLKLNSQKTLSSNTVVTHSIKPDKLYWITNQIKMEKLQDSLLFIHDLSCKYPNSGSLCRALDSFFDKISKVNKERDDIIVMISILVDIMLKNPRTYPIGAGILSKLISLIKPIDKQVEILKTIIYRFQKLPNIGYLEIWIQRIALKIDKEITFGELLCKKLNNKNSNISLWNSDWIANNKLKKIINDQLIVNEKTVENMSKVVKGNEVKLFYSKNNYYF

SEQ ID NO: 6 - DNA, 1461 bp, Synechococcus aeruginosa
  source 1..1461: mol_type = other DNA
ttgccaaagtacttcagctttaaggaacttcttgaggaaattgccaaagaagttaatgggaagaaactatctgactattatgggagtactatagactcagcaggaaaacaaaaactatcacgaccctgttactatgaaaatgttaaccatagatttctaaataataaagatggcaagtttgcttggagaccttttcaaataattcatccagctatctatgtatctcttgtaaatgaaattacatccgaaaataattggagagagattgttgctgcttttaagcgttttagctttaataatcagattaaatgcttatctattccaattgaaagcgaaaatttactatcagataaggctgaaaatataagcaattggtggcattcagttgagcaaaattcgattgagctatcgcttaagtatgagtatattatgcatacggatatttctgattgttatggatcaatatacactcattcaatagtatgggcattacatacaaagaaaattgctaaagaacaaagaagagatcgttctctaattggaaatataattgactcacatatacaagatatgtcatttgctcaaacaaatggaattcctcaaggtagtgggttgatggacttaatagctgaaattgttttgggctttgccgatcttgaattatcagaaaagttggaaagctatccacacttaatagattatcaaatcttaagatatagagatgattacagagtttttacaaataatccacaagaatctgacttaattgtaaagtccttaactgagatacttggtaatttggggttaaaacttaattcacaaaaaacattatcttccaatacagttgttactcactctattaagccagacaaattatattggataacaaatcagataaagatggaaaaattacaagattctttattatttatacatgatttatcctgtaaatatcctaactctggaagtctatgtagggcattagatagcttctttgacaaaattagtaaagtcaataaagaaagagatgatattatagttatgataagtattcttgttgatattatgctcaaaaatccaagaacttatcctattggggcaggaatactaagtaagttgatttctctgattaagccaatagataagcaggttgagattcttaaaactataatttatagatttcaaaagttacctaatataggttatcttgaaatttggattcagagaattgctttaaaaatcgataaggaaataacttttggcgagttattatgtaagaaactaaataataaaaattcaaatatttccttatggaattcagattggatagcaaataataaattaaaaaaaattattaatgatcaactaattgtgaacgagaagactgttgaaaatatgagtaaagtagttaaagggaatgaagtaaagcttttttattcaaaaaataattattatttctaa

SEQ ID NO: 7 - protein, 429 aa, Oenococcus oeni
MEMGQRMTNGDARKRRNLLDMSNAEARDFFLKPESYITFELPEYFSFDTVLTTASQSLEDASLASMTAKGKSLSNVADVNYKMLISKDGQFDWRPFQIIHPVTYVDLANCITEESNWKKIIKRFEEFEANPRIRCISIPVESLTSQKDTAATILNWWENLEQASIEYSLDYAYCIKTDITNCYGSIYTHSISWALHGKSWSKQHRKPSNGVGNRIDNKIQHLQFGQTNGIPQGSTLFDFVAEMVLGYSDLMLSNRLNEKKISNYQIIRFRDDYRIFSNSKSDCEQIAKELSDVLADLNMHFNSKKTMLTTDIISAAVKPDKSYWIKTSPLIWFKQGKNTYYRLSLQKHLMQIYEFSIKFPNSGSLKRALKEFLDRVSSLQKCPDDLEQLISIMSVLLLKNPIGVPLGVAILSRLFVFIDQKQVSSLLTQ

SEQ ID NO: 8 - DNA, 1290 bp, Oenococcus oeni
  source 1..1290: mol_type = other DNA
atggaaatgggtcaaagaatgactaacggtgatgcaagaaagagacgtaatttattagatatgtcgaacgctgaggcaagagatttttttctgaagcccgaaagttacattacttttgaattaccagaatattttagctttgacacagtcctgaccacggcaagtcaatctttggaagatgcttctctcgcaagcatgacggcgaagggcaaaagtctttctaatgtagctgatgttaattataaaatgctgataagtaaagatggccaattcgattggcggccctttcaaatcattcatccagtgacgtatgttgatttggctaactgcataacagaagagtcgaattggaagaaaataatcaaacgatttgaggaatttgaagcaaatcctagaattagatgtattagcattccggtggaatctcttacgagtcaaaaagatacagcagccacgattttaaattggtgggaaaatttggaacaagcctcaatcgaatatagcttggattatgcctactgtatcaaaacagatattactaattgctatggttcaatctatacacattcaatttcttgggcattgcatggcaagagctggtctaaacagcataggaagccctcaaatggagttggtaaccgaattgacaacaagatacagcatttacaatttggacaaacgaatgggattccacaagggagtacgctgtttgattttgtagcagaaatggttttgggatattccgatctgatgttatccaatcgtctcaatgagaaaaaaattagcaattatcagattattcgtttcagggacgattaccgaattttttctaattcgaaaagcgattgtgaacagattgctaaggaactctccgatgtgcttgctgatctcaatatgcattttaattcgaaaaaaacaatgttgaccactgatattatttcagcggcagttaaaccagataaaagttattggataaaaactagtcccttgatctggttcaaacaaggcaaaaatacatactatcgcttaagtttgcagaaacatttaatgcagatttatgagttttcaataaaatttccgaattccggaagcttgaaaagggctctgaaggaattcttggacagagtatcaagcttacagaaatgtccggacgacttagagcaacttattagtattatgtctgttcttttattgaaaaatcctatcggcgttcctttgggcgttgctatattgagccggttatttgtgtttattgatcagaaacaagtttcttctttgttgacgcaatga

SEQ ID NO: 9 - protein, 544 aa, Enterobacteria phage
MHFLNCSHSHRYHDRKQVVICAQLKKKSLIMKKIYELTTGRALKYFLQHDSYTTLELPSYVDFSSLLEEINSAIDEGKINFQPDSKSLMGKNINYEVLVSKDGLYSWRRITLINPLYYVYFCKLITSPSNWKAIRNKFREFESNDLFLCSSTPVSKKNTSNVAASVLNWWEDFEQKSLSLALEYEFMFSTDISNFYPSIYTHSFEWVFISKEEAKKKENNNNPGRLIDTHIQMMMSNQTNGIPLGSTLMDTFAELILGEIDLQLRKKTEEQKITDYKVVRYRDDYRIFSSSKDDLDKISKCLVEVLGEFGLDLNSRKTELHDDIILHSLKSAKKEYIIERSFNSLQKMLYAIYLFSLKHQNSKITVRYLNDFLRKLFRKKKITNSGHQLDAMLGIISSIMAKNPTTYPVGMAIFAKLLTFLYDDDELKFGKLQQLHCKLGKQPNTEMLDIWFQRVQGKIHTQWEGDYKTALCQRINDELKEKKTFTIDGLWDVEWIPGSAKNKNKQKILSILKKTKIVDLDAFEEMDNDITPEEVNLFDKEHSA

SEQ ID NO: 10 - DNA, 1635 bp, Enterobacteria phage
  source 1..1635: mol_type = other DNA
atgcacttcctgaattgctcgcattcgcatcgttatcatgatagaaaacaggtagttatttgtgcgcaacttaagaagaaatcactaattatgaaaaagatttatgaattaactactggtagagcgcttaagtattttctgcagcatgattcatacactactctggagctacccagttatgtcgatttttcttccttgcttgaagaaatcaactccgcgatagatgaaggtaaaatcaacttccaacctgactccaagtcattgatggggaagaatataaattacgaggttttagtcagcaaagatggcttatatagctggcgacgaataacactaattaatcctctgtactacgtgtatttttgtaaacttattacatccccttccaactggaaggctataaggaataaatttagagagtttgagtctaatgatctttttttatgctcaagtaccccagtgagcaaaaagaacacctcaaacgtagctgcatctgttttaaactggtgggaagattttgaacaaaaaagtctttcattggctcttgagtatgagttcatgttcagcacagatatttcaaacttctacccttctatttacactcatagctttgaatgggtattcatctcaaaagaagaggccaaaaagaaagaaaataacaataacccaggacgattgattgacactcatatccaaatgatgatgagtaatcagacaaacggaataccattgggtagtacgttgatggatacatttgccgaattaattttaggcgaaattgatttacagctaagaaaaaaaactgaagagcaaaaaataacggattacaaagtagttcgctacagagatgattatcgaatattttcaagcagtaaagatgatttggacaaaatctcaaagtgtttggttgaggtcttaggtgagtttgggcttgatttaaactcaagaaaaacagaactacatgacgacatcattcttcactcccttaaatcagcaaaaaaagaatatattatagaaaggtcgttcaactccctacagaaaatgttgtatgcaatatatttattttctttaaaacatcaaaactccaaaattacagttagatatttaaatgatttcttgcgaaaattatttaggaagaaaaagatcacaaatagtgggcatcaactagatgcaatgcttgggattatttcaagcatcatggctaaaaacccaaccacctacccagtgggaatggctattttcgcaaaactcttgaccttcctttatgacgacgatgaactcaaatttggcaagttacaacaacttcattgtaaactaggtaaacaaccaaatactgaaatgttagatatttggtttcaacgagttcaagggaagatacacacacaatgggaaggcgattacaaaacagccctatgtcaacgcataaatgatgaactcaaggaaaaaaaaacatttaccattgatggcctgtgggatgtagagtggattccgggttcagccaaaaataaaaacaaacagaaaatactatcaattttgaaaaaaacaaaaattgtggatttagatgcattcgaagaaatggataatgatattacaccagaagaagtgaacttattcgacaaggaacacagcgcttaa

SEQ ID NO: 11 - protein, 589 aa, Listeria monocytogenes
MIMRKKVDVLSMDNEKAREFFMKPSSYFSQNLPKYFNLGYLLESAESKLGKKQLKKEMYFENKVYSNFPNVNFLIQTNKTISTYRPITLLHPYIYVDYVNFLTEIKIWEELKERFQNLQEEVKEKIICSSLPFDIETSKEDDSKKEMALNFWKSIEQETIKYSLGYNYLLKLDISNFYGSIYTHTLCWAFHGENYSKEAKNSKNLQNLTGDKCDRKFQWMNYGETVGIPQGNVISDLMSELLLAYIDSELVKKIDDEIDYKIIRYRDDYRIFTKRLEDSTLVKRELVVLLQRFKLNLGESKTSQTTDIISGSLKEDKMYWIEHDPVTKLTSDKFYRSPKILMKKSLEEHKNKKYKTRVFNEFFKKHFHNRTYQATLQKHLLIIKVFSDLYPNSGQLIAALHEFEGRLLDLNYKDFRNIGTEVEVLIAILVDIIKKNPKITEIGVKLLSTLLKKINFEEFEMKYLESNNSNEVQSDFEVKLAYINSVNHKLSNSNYNDYLEIWMQRLVVKNLKESTTLSDLYIEQSKNELVKFCNSIIRGKEMIQIFNEDWLKDEHKIELSKFINLNEIDKLTDIISDDEIYLAEYNQMT

SEQ ID NO: 12 - protein, 610 aa, Staphylococcus aureus
MVNLKIRNLYNTILDSSFLIRYGYYDVRMKKIESKNINNEELEDYYGKPDTFYLKIFGLQDLYFLMKSEDLRGFFNFSDFKKNVDTEPIYFNTPKNNYVRREYKMPNVYSYLHLCFFIEDNKEEFINIFENNVQSTSKYFNELNFNFKFTKKIEQRLLFGGNSILSLDLSNFYHTLYTHSIPWVIHGKQNSKDNRYKGFANNLDSLIQKCQYGETHGIPVGNIISRIIAELYMCYIDKKLIEKGYKYARYVDDIKYPFVSNTDKEGFLMEFNSICREYNLILNDKKTDVQTFPYRNNMQKVEIFSYLDSLNKSSEIADWKSKINDFIDFCLSEEVSGNKGAVKCIYSVVINKLRDSKMSSNKINNILVSREKLTKYNLYEKFLDISLKDSRLTNKFINFTEQLIDLKISKEKLKKIARQYFKENKEIWKGNLDYYILNGWNQEVYQILLYSVLFDEEKILNKKSLQLILKNDLDDYSKCLSVILWIKKKFSFKVLLNTLEDKLKEVHSSYNDDASVRMQEKYWLLRYFIFYINKNNIIDDAFFKKHYNENNIKKDRNNIIKSELNMHYVLKSDSRKKQYVNRVNEFFGFLLNNNVALIQTNYNHKLFEYL

SEQ ID NO: 13 - DNA, 1833 bp, Staphylococcus aureus
  source 1..1833: mol_type = other DNA
atggtgaatttaaaaattcgtaatttatataacacgattttagatagttcttttctaattaggtatggatactatgatgttagaatgaaaaagattgaatcaaaaaatattaataatgaagaattagaagattattatggcaaaccagatactttctatttaaaaatttttggtttgcaagacttatattttctgatgaaatcagaggatttaagaggattttttaatttctctgattttaaaaagaatgtagatacggaaccaatttattttaatactcctaaaaataattatgttcgtagagaatataagatgccaaatgtctatagttacttacatctatgtttttttatagaagataataaagaggaatttatcaatattttcgaaaacaatgtacaatcaacttcaaaatattttaatgaattaaactttaattttaaattcactaagaaaatagagcagaggcttctttttggagggaatagtattttgtctttagatttatcaaacttttatcatactttgtacacacatagtataccttgggtcatacacggaaaacagaactcgaaagataatagatataaagggtttgctaataatttggatagtttaattcaaaaatgtcaatatggagaaactcatggtatacccgtggggaatattatatcaaggataatagcagaactatatatgtgctatatagacaaaaaattaattgaaaaaggatataaatatgctagatatgtagatgatattaaatatccatttgtatcaaatacagataaagaaggatttttgatggaatttaattccatatgtcgagagtacaatttaattctaaatgataaaaaaacagatgtgcagacttttccatatcgtaataacatgcaaaaagtagagatttttagttatctagatagcttgaataaaagctcggaaattgcggattggaaatcaaaaattaatgattttattgatttctgcttaagtgaagaagtttcaggtaataaaggagcagtaaaatgcatttattcggtggtaataaataaacttagagattcaaaaatgtcctcaaataagataaacaatattcttgttagtagggaaaaattaacaaaatataacctatatgagaaatttttggatatttcattgaaagattcgaggttaacaaataaatttataaactttacagagcaattgatagacttaaaaattagtaaagaaaaactaaagaaaatagcaaggcaatattttaaagaaaacaaggaaatatggaaaggaaatttagattattatattttaaatggttggaatcaagaagtataccaaatattattatactctgttttattcgatgaagaaaaaatactaaataaaaaatctttacaattaatattaaaaaatgatttagatgattactcaaaatgtttgagtgtcattttgtggattaaaaagaaatttagctttaaagtgttgttaaatacattggaagataaattaaaagaagtgcattcttcttacaacgatgatgcaagcgttagaatgcaagaaaaatattggttattaaggtactttattttttatataaataaaaataatattattgacgatgcatttttcaaaaaacattacaatgaaaataatataaaaaaagataggaacaatatcataaagtctgaacttaatatgcactatgttttaaaaagtgattcgagaaaaaagcaatatgtaaacagagtgaatgagttttttggatttttattaaataataatgtggcattaattcagacaaactataaccataaattatttgaatatctataa

SEQ ID NO: 14 - protein, 10 aa, synthetic construct
EQKLISEEDL

SEQ ID NO: 15 - protein, 9 aa, synthetic construct
YPYDVPDYA

SEQ ID NO: 16 - protein, 218 aa, synthetic construct
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPK

SEQ ID NO: 17 - protein, 7 aa, synthetic construct
DYKDDDD

SEQ ID NO: 18 - protein, 26 aa, synthetic construct
KRRWKKNFIAVSAANRFKKISSSGAL

SEQ ID NO: 19 - protein, 15 aa, synthetic construct
KETAAAKFERQHMDS

SEQ ID NO: 20 - protein, 14 aa, synthetic construct
GKPIPNPLLGLDST

SEQ ID NO: 21 - protein, 6 aa, synthetic construct
HHHHHH

SEQ ID NO: 22 - protein, 599 aa, Lactococcus lactis
MKKEFTELYDFIFDPIFLVRYGYYDISIPNKKMNTEKVEIDNDYGKSDSFYFKVFNMESLANYLRSHDLKKHFNGNKPLSTEPVYFNIPKNIEARRQYKMPNLYSYMALNYYICDNKKEFIDVFMDNKFSTSKFFNQLSFDYPKTQEIRQTLLYGGIKKLHLDLSNFYHTLYTHSIPWMIDGKSTAKKNRKKGFSNRLDTLITACQYEETHGIPTGNLLSRIITELYMCHFDKQMERKNFVYSRYVDDFIFPFTLENEKQEFLNEFNLICRENNLLINDNKTKVDNFPFVDQSSKSDIFSFFENITSMNSNDKWIKEISDFIDYCVNEEHLGNKGAIKCIFPVIKNTLKQKKVDTKNIDIIFSKRNMVTNFNVFEKILDLSLKDSKLTNKFLTFFENINEFGFSSLSASNIVKKYFSNNSKGIKNKIDHYRKNNFNQELYQILLYAVVFEIDDLLNQEELLNLIDSNIDDYSLILGTILYLKNSSYKLDKLLKKIDSLFINTHANYNVNTSRMAEKLWLFRYFFYFLNCKDIISKIEINSYCKSKKYKSGPNGYQTELNWNYIKGQGNDLRANDFFNELILAEVWLIYCGENEDFKYLN

SEQ ID NO: 23 - protein, 606 aa, Enterococcus faecalis
MKKEFTDMKKEFTELYDFIFDPIFLVRYGYYDISIPNKKMNTEKVEIDNDYGKSDSFYFKVFNMESLANYLRSHDLKKHFNGNKPLSTEPVYFNIPKNIEARRQYKMPNLYSYMALNYYICDNKKEFIDVFMDNKFSTSKFFNQLSFNYPKTQEIRQTLLYGGIKKLHLDLSNFYHTLYTHSIPWMIDGKSTAKKNRKKGFSNRLDTLITACQYEETHGIPTGNLLSRIITELYMCHFDKQMERKNFVYSRYVDDFIFPFTLENEKQEFLNEFNLICRENNLLINDNKTKVDNFPFVDQSSKSDIFSFFENITSMNSNDKWIKEISDFIDYCVNEEHLGNKGAIKCIFPVIKNTLKQKKVDTKNIDIIFSKRNMVTNFNVFEKILDLSLKDSKLTNKFLTFFENINEFGFSSLSASNIVKKYFSNNSKGIKNKIDHYRKNNFNQELYQILLYAVVFEIDDLLNQEELLNLIDSNIDDYSLILGTILYLKNSSYKLDKLLKKIDSLFINTHANYNVNTSRMAEKLWLFRYFFYFLNCKDIISKIEINSYCKSKKYKSGPNGYQTELNWNYIKGQGNDLRANDFFNELILAEVWLIYCGENEDFKYLN

SEQ ID NO: 24 - protein, 599 aa, Vagococcus fluvialis
MKKEFTELYDFIFDPIFLVRYGYYDISIPNKKMNTEKVEIDNDYGKSDSFYFKVFNMESLANYLRSHDLKKHFNGNKPLSTEPVYFNIPKNIEARRQYKMPNLYSYMALNYYICDNKKEFIDVFMDNKFSTSKFFNQLSFDYPKTQEIRQTLLYGGIKKLHLDLSNFYHTLYTHSIPWMIDGKSTAKKNRKKGFSNRLDTLITACQYEETHGIPTGNLLSRIITELYMCHFDKQMERKNFVYSRYVDDFIFPFTLENEKQEFLNEFNLICRENNLLINDNKTKVDNFPFVDQSSKSDIFSFFENITSMNSNDKWIKEISDFIDYCVNEEHLGNKGAIKCIFPVIKNTLKQKKVDTKNIDIIFSKRNMVTNFNVFEKILDLSLKDSKLTNKFLTFFENINEFGFSSLSASNIVKKYFSNNSKGIKNKIDHYRKNNFNQELYQILLYAVVFEIDDLLNQEELLNLIDSNIDDYSLILGTILYLKNSSYKLDKLLKKIDSLFINTHANYNVNNSRMAEKLWLFRYFFYFLNCKDIISKIEINSYCKSKKYKSGPNGYQTELNWNYIKGQGNDLRANDFFNELILAEVWLIYCGDNEDFKYLN

SEQ ID NO: 25 - protein, 600 aa, Lactococcus lactis
MESVFTELYDLMFDPVFLVRYGYYDITIKNKKMNTEKVEIENDYGKSDSFYFKVFNMESFSEYLRGHDLKKFFKYGKPISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASDIVKKYFSVNSNSIGRKIDYYHKNHFNQELYQILLYAVEFEIDNLLTQEELLKLINSNTDDFSLVLVTILYLKNSSYKRNELLEKIDSLFIDTHVNYPSDTARMSEKFWLFRYFFYFLQSKENINKKEVNTYCKSKNYNIGKNGYESELNWRYIRGSASNTSVNDFFNELIENEVWLIYCGENGDFKYLPR

SEQ ID NO: 26 - protein, 600 aa, Lactococcus cremoris
MESVFTELYDLIFDPVFLVRYGYYDITIKNKKMNTEKVEIENDYGKSDSFYFKVFNMESFSEYLRGHDLKNFFKYEKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMMTGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASDIVKKYFSVNSKSIGRKIDYYHKNHFNQELYQILLYAVEFEIDNLLTQEELLKLITTNTDDFSLVLVTILYLKNGSYKRNELLEKIDSLFIDTHVNYPSDTARMSEKFWLFRYFFYFLQSKENINKKEVNTYCKSKNYNIGKNGYESELNWRYIRGSASNTSVNDFFNELIENEVWLIYCGENGDFKYLPR

SEQ ID NO: 27 - protein, 600 aa, Lactococcus petaurid
MESVFTELYDLIFDPVFLVRYGYYDITIKNKKMNTEKVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMMTGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASDIVKKYFSVNSKSIGRKIDYYHKNHFNQELYQILLYAVEFEIDNLLTQEELLKLIATNTDDFSLVLVTILYLKNGSYKRNELLEKIDSLFIDTHVNYPSDTARMSEKFWLFRYFFYFLQSKENINKKEVNTYCKSKNYNIGKNGYESELNWRYIRGSASNTSVNDFFNELIENEVWLIYCGENGDFKYLPR

SEQ ID NO: 28 - protein, 600 aa, Lactococcus lactis
MESVFTELYDLIFDPVFLVRYGYYDITIKNKKMNTEKVEIENDYGKSDSFYFKVFNMESFSEYLRGHDLKNFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMMTGKAEAKKDRKNGFANTLDKLITSCQYDETHGIPTGNLLSRIIAELYMCHFDKRMENNNFIYTRYVDDIVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASDIVKKYFSVNSKSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLQSKENINKKEVNTYCKSKNYNIGKNGYESELNWRYIRGSASNTSVNDFFNELIENEVWLIYCGGNGDFKYLPR

SEQ ID NO: 29 - protein, 600 aa, Lactococcus petaurid
MESVFTELYDLIFDPVFLVRYGYYDITIKNKKMNTEKVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGKNGYESELNWRYIRGSASNTSVNDFFNELIENEVWLIYCGENGDFKYLPR

SEQ ID NO: 30 - protein, 600 aa, Lactococcus lactis
MESVFTELYDLIFDPVFLVRYGYYDITIKNKKMNTEKVEIENDYGKSDSFYFKVFNMESFSEYLRGHDLKNFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGKNGYESELNWRYIRGSASNTSVNDFFNELIENEVWLIYCGENGDFKYLPR

SEQ ID NO: 31 - protein, 600 aa, Lactococcus cremoris
MESVFTELYDLIFDPVFLVRYGYYDITIKNKKMNTEKVEIENDYGKSDSFYFKVFNMESFSEYLRGHDLKNFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMMTGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITNFNIFEKILDLSLKDSRLTNKFLTFFENMSSLGFSSISASEIVKKYFSANSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGNNGYESELNWRYIRGSASITSVNDFFNELIENEVWLIYCGENGDFKYLPR

SEQ ID NO: 32 - protein, 600 aa, Lactococcus cremoris
MESVFTELYDLIFDPVFLVRYGYYDITIKNKKMNTEKVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMMTGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITNFNIFEKILDLSLKDSRLTNKFLTFFENMSSLGFSSISASEIVKKYFSANSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGNNGYESELNWRYIRGSASITSVNDFFNELIENEVWLIYCGENGDFKYLPR

SEQ ID NO: 33 - protein, 600 aa, Lactococcus petaurid
MESVFTELYDLIFDPVFLVRYGYYDITIKNKKMNTEKVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCYFDKRMENNNFIYTRYVDDVVFPFTLEAEKEDFLKEFSLICRENNLLVNDNKTRVNNFPFTNKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSSSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLQSKENINKKEVNTYCKSKNYNIGKNGYKSELNWRYIRGSASNTSVNDFFNELIENEVWLIYCGENGDFKYLPR

SEQ ID NO: 34 - protein, 564 aa, Lactococcus petaurid
KVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGKNGYESELNWRYIRGSASNTSVNDFFNELIENEVWLIYCGENGDFKYLPR

SEQ ID NO: 35 - protein, 551 aa, Lactococcus garvieae
FYFKVFNMESFSEYLRGHDLKKFFKYGKSISTEPVYFSIPKNINNRRQYKMPNLYSYMALNYYMCDQKKEFVDVFVSNKFSTSKFFNQLNFDYSTTQEISQTLLYGGVKKLYLDLSNFYHTLYTHSIPWMITGKADAKKDRKNGFANTLDKLITSCQYDETHGIPTGNLLSRIIAELYMCYFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSSSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLQSKENINKKEVNTYCKSKNYNIGKNGYESELNWRYIRGSASNTSVNDFFNELIENEVWLIYCGENGDFKYLPR

SEQ ID NO: 36 - protein, 529 aa, unidentified
MESVFTELYDLMFDPVFLVRYGYYDITIKNKKMNTEKVEIENDYGKSDSFYFKVFNMESFSEYLRGHDLKKFFKYGKPISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASDIVKKYFSVNSNSIGRKIDYYHKNHFNQELYQILLYAVEFEIDNLLTQEELLKLINSNTDDFSLVLVTILYLKNSSYKRNELLEKIDSLFIDTHVNYPSDTARMSEKFWLFRYFFYFLHK

SEQ ID NO: 37 - protein, 476 aa, Lactococcus petaurid
MESVFTELYDLIFDPVFLVRYGYYDITIKNKKMNTEKVEIENDYGKSDSFYFKVFNMESFSEYLRGHDLKKFFKYGKSISTEPVYFSIPKNINSRRQYKMPNLYSYMALNYYMCDQKKEFVDVFVSNKFSTSKFFNQLNFDYSTTQEISQTLLYGGVKKLYLDLSNFYHTLYTHSIPWMITGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITDFNIFEKILDLSLKDSRLTNKFLTFFENMSSLGFSSISASEIVKKYFSVNSNSIGRKIDYYHKNHFNQELYQILLYAVEFEIDNLLTQEELLKLITSNTDDFSLVLV

SEQ ID NO: 38 - protein, 372 aa, Streptococcus parauberis
MCHFDKQMERKNFVYSRYVDDFIFPFTLENEKQEFLNEFNLICRENNLLINDNKTKVDNFPFVDQSSKSDIFSFFENITSMNSNDKWIKEISDFIDYCVNEEHLGNKGAIKCIFPVIKNTLKQKKVDTKNIDIIFSKRNMVTNFNVFEKILDLSLKDSKLTNKFLTFFENINEFGFSSLSASNIVKKYFSNNSKGIKNKIDHYRKNNFNQELYQILLYAVVFEIDDLLNQEELLNLIDSNIDDYSLILGTILYLKNSSYKLDKLLKKIDSLFINTHANYNVNTSRMAEKLWLFRYFFYFLNCKDIISKKEINSYCESKKYKSGPNGYQTELNWNYIKGQGNDLRANDFFNELILAEVWLIYCGENEDFKYLN

SEQ ID NO: 39 - protein, 422 aa, Lactococcus cremoris
MMTGKAEAKKDRKNGFANTLDKLITSCQYDETHGIPTGNLLSRIIAELYMCHFDKRMENNNFIYTRYVDDVVFPFTLEAEKEDFLKEFSLICRENNLLVNDNKTRVDNFPFINKSSKSNIFSFFENLTLKNSDEKWIKEISNFIDYCINEESLGNKGAIKSIFPVIKNTFKNKKISSAKLNNIFSKKDIITNFNIFEKILDLSLKDSRLTNKFLTFFENMSSLGFSSISASEIVKKYFSANSNSIGRKIDYYHKNHFNQELYQILLYAVEFEIDNLLTQEELLKLITSNTDDFSLVLVTILYLKNGSYKRNELLEKIDSLFIDTHVNYPSDTARMSEKFWLFRYFFYFLLSKENINKKEVNTYCKSKNYNIGNNGYESELNWRYIRGSASITSVNDFFNELIENEVWLIYCGENGDFKYLPR

SEQ ID NO: 40 - protein, 387 aa, synthetic construct
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 41 - protein, 283 aa, synthetic construct
MAEITASLVKELRERTGAGMMDCKKALTEANGDIELAIENMRKSGAIKAAKKAGNVAADGVIKTKIDGNYGIILEVNCQTDFVAKDAGFQAFADKVLDAAVAGKITDVEVLKAQFEEERVALVAKIGENINIRRVAALEGDVLGSYQHGARIGVLVAAKGADEELVKHIAMHVAASKPEFIKPEDVSAEVVEKEYQVQLDIAMQSGKPKEIAEKMVEGRMKKFTGEVSLTGQPFVMEPSKTVGQLLKEHNAEVTGFIRFEVGEGIEKVETDFAAEVAAMSKQS